(19) United States
(12) Patent Application Publication
(10) Pub. No.: US 2016/0194673 A1
(43) Pub. Date: Jul. 7, 2016

(54) PRODUCTION METHOD FOR GLYCEROL PHOSPHATE

(71) Applicant: THERMOSTABLE ENZYME LABORATORY CO., LTD., Kobe-shi., Hyogo (JP)

(72) Inventors: Hideyuki Iwata, Hyogo (JP); Hisae Tanaka, Hyogo (JP)

(21) Appl. No.: 14/769,012

(22) PCT Filed: Feb. 19, 2014
(86) PCT No.: PCT/JP2014/053888

§ 371 Date: Nov. 25, 2015

(30) Foreign Application Priority Data
Feb. 19, 2013 (JP) 2013-029701

(51) Int. Cl. C12P 9/00

(52) U.S. Cl. CPC C12P 9/00

(57) ABSTRACT

The present invention addresses the problem of providing a method for the efficient phosphorylation of glycerol. The problem is solved by reacting glycerol with either a kinase that includes the active center expressed by sequence (1) and exhibits a catalytic activity with respect to the phosphorylation of glycerol, or a kinase that includes the amino acid sequence represented by SEQ ID NO: 2 and exhibits a catalytic activity with respect to the phosphorylation of glycerol, in the presence of a phosphate group donor.

### TECHNICAL FIELD

The present invention relates to a method for the production of a phosphorylated glycerol with high efficiency.

### BACKGROUND ART

Since phosphorylated glycerol is highly useful, for example, as raw material for cosmetics and as intermediates for producing pharmaceuticals, methods have been investigated in which a phosphorylated glycerol can be obtained with high efficiency. A method for achieving efficient phosphorylation is, for example, one involving the use of a phosphorylating enzyme that catalyzes the phosphorylation of glycerol. Many types of phosphorylating enzymes have been known until now, and there have been found those derived from many different species (Non-Patent Document 1). However, even though any of these many phosphorylating enzymes which exist is employed, the improvement in the efficiency of the phosphorylation reaction is not always accomplished. In addition, it is necessary that in reactions for obtaining a phosphorylated glycerol, a phosphorylating enzyme and glycerol are reacted in the presence of a phosphate group donor. Phosphorylating enzymes that have been used in the past, however, have a problem that expensive ATP (adenosine triphosphate) is required as a phosphate group donor in the reaction, thereby increasing production costs. Polyphosphoric acids, on the other hand, are mentioned as an example of an inexpensive phosphate group donor, but there have not been known any enzymes that use a polyphosphoric acid to catalyze the glycerol phosphorylating reaction (see, for example, Non-Patent Document 2). Because of these backgrounds, there is a great need for a method by which a phosphorylated glycerol of great industrial usefulness is produced with high efficiency and at low cost.

### PRIOR ART DOCUMENTS

### Non-Patent Documents

Non-Patent Document 1: Cell Mol Life Sci., 1998 August; 54(8): 833-50.
Non-Patent Document 2: Protein Eng., 1998 December; 11(12): 1219-27.

### SUMMARY OF THE INVENTION

### Problems to be Solved by the Invention

The present invention has a main purpose of providing a method for the phosphorylation of glycerol in which a high efficiency of phosphorylation can be achieved.

### Means for Solving the Problem

The inventors have made intensive studies to solve the above problem, with the result that it has been found that the efficiency of the phosphorylation of glycerol is significantly improved by using a phosphorylating enzyme derived from a particular microorganism. In addition, it has been found that by using this enzyme, a phosphorylated glycerol is efficiently obtained even when the phosphorylation reaction is performed using an inexpensive phosphate group donor like a polyphosphoric acid. Further, the inventors have identified the amino acid sequence responsible for the activity of the phosphorylating enzyme obtained from the particular microorganism. The present invention has been completed as a result of further improvements based on these findings.

Thus, the present invention provides methods for phosphorylation according to the embodiments described below:

Embodiment 1: a method for the production of a phosphorylated glycerol, comprising a step of allowing a phosphorylating enzyme to act on glycerol in the presence of a phosphate group donor,

wherein the phosphorylating enzyme is a class A acid phosphatase and includes a polypeptide comprising as an active center an amino acid sequence represented by the sequence (1):

-X¹-X²-X³-Pro-Ser-Gly-His-X⁴- (1)

wherein,
X¹ denotes glycine, alanine, or phenylalanine;
X² denotes any amino acid;
X³ denotes tyrosine or tryptophan; and
X⁴ denotes threonine, serine, or alanine;
with the proviso that when X¹ is phenylalanine, X⁴ is threonine or serine.

Embodiment 2: the method for production according to embodiment 1, wherein X¹ and

X⁴ in the sequence (1) are any of (A1) to (A8) below:
(A1) X¹ being glycine and X⁴ being threonine;
(A2) X¹ being glycine and X⁴ being serine;
(A3) X¹ being alanine and X⁴ being serine;
(A4) X¹ being alanine and X⁴ being threonine;
(A5) X¹ being alanine and X⁴ being alanine;
(A6) X¹ being glycine and X⁴ being alanine;
(A7) X¹ being phenylalanine and X⁴ being threonine; and
(A8) X¹ being phenylalanine and X⁴ being serine.

Embodiment 3: the method for production according to embodiment 1 or 2, wherein in the sequence (1), X² is serine, alanine, or aspartic acid.

Embodiment 4: the method for production according to embodiment 1 or 3, wherein X¹, X², X³, and X⁴ in the sequence (1) are any of (B1) to (B11) below:

(B1) X¹ being glycine, X² being serine, X³ being tyrosine, and X⁴ being threonine;
(B2) X¹ being glycine, X² being alanine, X³ being tyrosine, and X⁴ being threonine;
(B3) X¹ being glycine, X² being serine, X³ being tyrosine, and X⁴ being serine;
(B4) X¹ being glycine, X² being aspartic acid, X³ being tyrosine, and X⁴ being
threonine;
(B5) X¹ being alanine, X² being serine, X³ being tyrosine, and X⁴ being serine;
(B6) X¹ being alanine, X² being serine, X³ being tyrosine, and X⁴ being threonine;
(B7) X¹ being alanine, X² being serine, X³ being tyrosine, and X⁴ being alanine;
(B8) X¹ being glycine, X² being serine, X³ being tyrosine, and X⁴ being alanine;
(B9) X¹ being phenylalanine, X² being serine, X³ being tyrosine, and X⁴ being
threonine;
(B10) X¹ being phenylalanine, X² being serine, X³ being tyrosine, and X⁴ being serine;
and
(B11) X¹ being glycine, X² being serine, X³ being tryptophan, and X⁴ being serine.

Embodiment 5: the method for production according to any of embodiments 1 to 4, wherein the histidine (His) residue at the 7-th position in the amino acid sequence represented by the sequence (1) is located between positions 120 and 160 from the N-terminus of the polypeptide employed as the phosphorylating enzyme.

Embodiment 6: the method for production according to any of embodiments 1 to 5, wherein the phosphorylating enzyme further includes a polypeptide as defined in (I) or (II) below:

including a polypeptide as defined in (II);
(I) a polypeptide that has the amino acid sequence set forth in SEQ ID NO: 2, on the N-terminal side of a stretch of the amino acid sequence coding for the active center represented by the sequence (1), or
(II) a polypeptide that comprises, on the N-terminal side of a stretch of the amino acid sequence coding for the active center represented by the sequence (1), an amino acid sequence having one or a few amino acids substituted, deleted, inserted, or added in the amino acid sequence set forth in SEQ ID NO: 2 and has an activity to catalyze the glycerol phosphorylating reaction which is comparable or superior to that of the polypeptide as defined in (I).

Embodiment 7: the method for production according to embodiment 6, wherein the amino acid residue at the first position in the amino acid sequence set forth in SEQ ID NO: 2 is located between positions 5 and 40 from the N-terminus of the polypeptide employed as the phosphorylating enzyme.

Embodiment 8: the method for production according to any of embodiments 1 to 5, wherein the phosphorylating enzyme further includes any of the polypeptides as defined in (III) to (VI) below:

(III) a polypeptide that has the amino acid sequence set forth in any of SEQ ID NOs: 26 to 32 and 106 to 110, on the N-terminal side of the amino acid sequence represented by the sequence (1);
(IV) a polypeptide that has, on the N-terminal side of the amino acid sequence represented by the sequence (1), an amino acid sequence having one or several amino acids substituted, deleted, inserted, or added in the amino acid sequence set forth in any of SEQ ID NOs: 26 to 32 and 106 to 110 and has an activity to catalyze the glycerol phosphorylating reaction which is comparable or superior to that of its corresponding polypeptide as defined in (III);
(V) a polypeptide that has the amino acid sequence set forth in any of SEQ ID NOs: 33 to 39 and 111 to 115, on the C-terminal side of the amino acid sequence represented by the sequence (1); and
(VI) a polypeptide that has, on the C-terminal side of the amino acid sequence represented by the sequence (1), an amino acid sequence having one or several amino acids substituted, deleted, inserted, or added in the amino acid sequence set forth in any of SEQ ID NOs: 33 to 39 and 111 to 115 and has an activity to catalyze the glycerol phosphorylating reaction which is comparable or superior to that of its corresponding polypeptide as defined in (V).

Embodiment 9: the method for production according to any of embodiments 1 to 8, wherein the phosphorylating enzyme includes a polypeptide as defined in (VII) or (VIII) below:

(VII) a polypeptide that has the amino acid sequence set forth in any of SEQ ID NOs: 3 to 9, 13, 104, 105, 116, 117, and 119 to 121, or
(VIII) a polypeptide that includes an amino acid sequence having, in the amino acid sequence set forth in any of SEQ ID NOs: 3 to 9, 13, 104, 105, 116, 117, and 119 to 121, one or several amino acids substituted, deleted, inserted, or added in a region outside the active center including the amino acid sequence represented by the sequence (1) and has an activity to catalyze the glycerol phosphorylating reaction which is comparable or superior to that of its corresponding polypeptide as defined in (VII).

Embodiment 10: a method for the production of a phosphorylated glycerol, comprising a step of allowing a phosphorylating enzyme to act on glycerol in the presence of a phosphate group donor,

wherein the phosphorylating enzyme is a class A acid phosphatase and includes a polypeptide as defined in (i) or (ii) below:
(i) a polypeptide that has the amino acid sequence set forth in SEQ ID NO: 2 and has an activity to catalyze the glycerol phosphorylating reaction, or
(ii) a polypeptide that has an amino acid sequence having one or several amino acids substituted, deleted, inserted, or added in the amino acid sequence set forth in SEQ ID NO: 2 and has an activity to catalyze the glycerol phosphorylating reaction which is comparable or superior to that of the polypeptide as defined in (i).

Embodiment 11: the method for production according to embodiment 10, wherein the amino acid residue at the first position in the amino acid sequence set forth in SEQ ID NO: 2 is located between positions 5 and 40 from the N-terminus of the polypeptide employed as the phosphorylating enzyme.

Embodiment 12: the method for production according to embodiment 10 or 11, wherein the phosphorylating enzyme includes a polypeptide having, on the C-terminal side of the amino acid sequence set forth in SEQ ID NO: 2, an amino acid sequence represented by the sequence (1):

-X¹-X²-X³-Pro-Ser-Gly-His-X⁴- (1)

wherein,
X¹ denotes glycine, alanine, or phenylalanine;
X² denotes any amino acid;
X³ denotes tyrosine or tryptophan; and
X⁴ denotes threonine, serine, or alanine;
with the proviso that when X¹ is phenylalanine, X⁴ is threonine or serine.

Embodiment 13: the method for production according to embodiment 12, wherein X¹ and X⁴ in the sequence (1) are any of (A1) to (A8) below:

(A1) X¹ being glycine and X⁴ being threonine;
(A2) X¹ being glycine and X⁴ being serine;
(A3) X¹ being alanine and X⁴ being serine;
(A4) X¹ being alanine and X⁴ being threonine;
(A5) X¹ being alanine and X⁴ being alanine;
(A6) X¹ being glycine and X⁴ being alanine;
(A7) X¹ being phenylalanine and X⁴ being threonine; and
(A8) X¹ being phenylalanine and X⁴ being serine.

Embodiment 14: the method for production according to embodiment 12 or 13, wherein in the sequence (1), X² is serine, alanine, or aspartic acid.

Embodiment 15: The method for production according to any of embodiments 12 to 14,

wherein X¹, X², X³, and X⁴ in the sequence (1) are any of (B1) to (B11) below:
(B1) X¹ being glycine, X² being serine, X³ being tyrosine, and X⁴ being threonine;
(B2) X¹ being glycine, X² being alanine, X³ being tyrosine, and X⁴ being threonine;
(B3) X¹ being glycine, X² being serine, X³ being tyrosine, and X⁴ being serine;
(B4) X¹ being glycine, X² being aspartic acid, X³ being tyrosine, and X⁴ being threonine;
(B5) X¹ being alanine, X² being serine, X³ being tyrosine, and X⁴ being serine;
(B6) X¹ being alanine, X² being serine, X³ being tyrosine, and X⁴ being threonine;
(B7) X¹ being alanine, X² being serine, X³ being tyrosine, and X⁴ being alanine;
(B8) X¹ being glycine, X² being serine, X³ being tyrosine, and X⁴ being alanine;
(B9) X¹ being phenylalanine, X² being serine, X³ being tyrosine, and X⁴ being threonine;
(B10) X¹ being phenylalanine, X² being serine, X³ being tyrosine, and X⁴ being serine; and
(B11) X¹ being glycine, X² being serine, X³ being tryptophan, and X⁴ being serine.

Embodiment 16: the method for production according to any of embodiments 12 to 15, wherein the histidine (His) residue at the 7-th position in the amino acid sequence represented by the sequence (1) is located between positions 120 and 160 from the N-terminus of the polypeptide employed as the phosphorylating enzyme.

Embodiment 17: the method for production according to any of embodiments 10 to 16, wherein the phosphorylating enzyme further comprises at least one of the amino acid sequences as defined in (iii) to (vi) below:

(iii) a polypeptide that has, on the N-terminal side of the amino acid sequence set forth in SEQ ID NO: 2, the amino acid sequence set forth in SEQ ID NO: 40 and has an activity to catalyze the glycerol phosphorylating reaction;
(iv) a polypeptide that has, on the N-terminal side of the amino acid sequence set forth in SEQ ID NO: 2, an amino acid sequence having one or a few amino acids substituted, deleted, inserted, or added in the amino acid sequence set forth in SEQ ID NO: 40 and has an activity to catalyze the glycerol phosphorylating reaction which is comparable or superior to that of the polypeptide as defined in (iii);
(v) a polypeptide that has, on the C-terminal side of the amino acid sequence set forth in SEQ ID NO: 2, the amino acid sequence set forth in SEQ ID NO: 41 and has an activity to catalyze the glycerol phosphorylating reaction; and
(vi) a polypeptide that has, on the C-terminal side of the amino acid sequence set forth in SEQ ID NO: 2, an amino acid sequence having one or a few amino acids substituted, deleted, inserted, or added in the amino acid sequence set forth in SEQ ID NO: 41 and has an activity to catalyze the glycerol phosphorylating reaction which is comparable or superior to that of the polypeptide as defined in (v).

Embodiment 18: the method for production according to any of embodiments 10 to 17, wherein the phosphorylating enzyme includes a polypeptide as defined in (vii) or (viii) below:

(vii) a polypeptide that has the amino acid sequence set forth in any of SEQ ID NOs: 9, 15 to 17, 19, 22, and 25; or
(viii) a polypeptide that includes an amino acid sequence having, in the amino acid sequence set forth in any of SEQ ID NOs: 9, 15 to 17, 19, 22, and 25, one or several amino acids substituted, deleted, inserted, or added in a region outside the amino acid sequence set forth in SEQ ID NO: 2 and has an activity to catalyze the glycerol phosphorylating reaction which is comparable or superior to that of the polypeptide as defined in (vii).

Embodiment 19: the method for production according to any of embodiments 1 to 18, wherein the phosphate group donor is a polyphosphoric acid.

Embodiment 20: the method for production according to any of embodiments 1 to 19, wherein the phosphorylated glycerol is α-glycerophosphate.

Embodiment 21: the method for production according to any of embodiments 1 to 20, wherein the pH of the reaction solution is from 4 to 5 in the step of allowing the phosphorylating enzyme to act on the glycerol in the presence of the phosphate group donor.

Embodiment 22: the method for production according to any of embodiments 1 to 21, wherein the glycerol is added in an amount of 1000 to 50000 parts by weight per part by weight of the phosphorylating enzyme, in the step of allowing the phosphorylating enzyme to act on the glycerol in the presence of the phosphate group donor.

Embodiment 23: the method for production according to any of embodiments 1 to 22, wherein the concentration of the phosphate group donor in the reaction solution is from 2% to 10% by weight in the step of allowing the phosphorylating enzyme to act on the glycerol in the presence of the phosphate group donor.

### Advantages of the Invention

According to the methods for phosphorylation according to the present invention, it is possible to significantly enhance the efficiency of the phosphorylation of glycerol. In addition, it is possible to use an inexpensive polyphosphoric acid as a phosphate group donor, thereby to allow reducing production costs.

### BRIEF DESCRIPTION OF THE DRAWINGS

FIG. 1 represents graphs showing the results of the phosphorylation of glycerol in which phosphorylating enzymes derived from various microorganisms were used. In FIG. 1, graph (i) shows the results when the reaction was carried out using 4 M of glycerol and at a pH of 5.0, graph (ii) shows the results when using 4 M of glycerol and at a pH of 4.0, and graph (iii) shows the results when using 0.5 M of glycerol and at a pH of 4.5.

FIG. 2 represents graphs showing the results of glycerol phosphorylating reactions in which the type of phosphate group donor was changed.

FIG. 3 represents a graph showing the amount of the resulting glycerol-3-phosphate in which the glycerol phosphorylating reaction was performed using Mutant a.

FIG. 4 represents a graph showing the amount of the resulting glycerol-3-phosphate in which the glycerol phosphorylating reaction was performed using Mutants b to g.

FIGS. 5(i) and (ii) show the structures of phosphorylating enzyme mutants A to L in FIG. 3. In FIGS. 3 (i) and (ii), the mutated portion in the amino acid sequences of these mutants is underlined.

FIG. 6 represents a graph showing the amount of the resulting glycerol-3-phosphate in which the glycerol phosphorylating reaction was performed using Mutants A to L.

### EMBODIMENTS OF THE INVENTION

### 1. Phosphorylating Enzymes

The present invention provides a method for the production of a phosphorylated glycerol. The method is characterized by allowing a given phosphorylating enzyme to act on glycerol in the presence of a phosphate group donor. Hereinafter, a method for the production of a phosphorylated glycerol according to the present invention is sometimes referred to simply as “a method of the present invention.”

The glycerol to be phosphorylated in the present invention is represented by C₃H₈O₃ and is also referred to as 1, 2, 3-trihydroxypropane or glycerin.

The method of the present invention uses as the phosphorylating enzyme a class A acid phosphatase, which catalyzes a glycerol phosphorylating reaction, thereby to obtain a phosphorylated glycerol. Here, an acid phosphatase is a phosphomonoesterase having a function of attaching free phosphate groups from other molecules. In addition, class A is a classification of acid phosphatases and indicates that enzymes of class A belong to a group of enzymes having the property that the enzymatic activity is not inhibited by EDTA and by inorganic phosphates. Further, a class A acid phosphatase is defined as a generic term for enzymes having an amino acid sequence of KX₆RP-(X₁₂₋₅₄)-PSGH-(X₃₁₋₅₄)-SRX₅HX₃D wherein each X independently denotes any amino acid (The EMBO Journal, Vol 19, No. 11, pp 2412-2423).

Examples of the phosphorylating enzyme which can be used in the present invention include an enzyme according to embodiment (α), that is, a phosphorylating enzyme comprising the active center represented by the sequence (1) as indicated below, and an enzyme according to embodiment (β), that is, a phosphorylating enzyme comprising the amino acid sequence set forth in SEQ ID NO: 2. The following will describe each of these phosphorylating enzymes in detail.

(1-1) Phosphorylating Enzymes According to Embodiment (α)

An embodiment of the phosphorylating enzyme according to embodiment (α) which can be used in the method of the present invention includes a phosphorylating enzyme that comprises the active center including an amino acid sequence represented by the sequence (1) (SEQ ID NO: 1) as indicated below and has an activity to catalyze the glycerol phosphorylating reaction:

-X¹-X²-X³-Pro-Ser-Gly-His-X⁴- (1)

wherein,
X¹ denotes glycine, alanine, or phenylalanine;
X² denotes any amino acid;
X³ denotes tyrosine or tryptophan; and
X⁴ denotes threonine, serine, or alanine;
with the proviso that when X¹ is phenylalanine, X⁴ is threonine or serine.

X² in the sequence (1) may be any amino acid and preferably is an uncharged amino acid, a non-polar amino acid, or an acidic amino acid, further preferably serine, alanine, or aspartic acid, with serine being particularly preferable.

A specific embodiment of the phosphorylating enzyme which can be used in the method of the present invention is an enzyme in which X¹, X², and X⁴ in the sequence (1) are any of (A1) to (A8) below:

(A1) X¹ being glycine and X⁴ being threonine; preferably X¹ being glycine, X² being serine, alanine, or aspartic acid, and X⁴ being threonine; further preferably X¹ being glycine, X² being alanine, and X⁴ being threonine;
(A2) X¹ being glycine and X⁴ being serine; preferably X¹ being glycine, X² being serine, and X⁴ being serine;
(A3) X¹ being alanine and X⁴ being serine; preferably X¹ being alanine, X² being serine, and X⁴ being serine;
(A4) X¹ being alanine and X⁴ being threonine; preferably X¹ being alanine, X² being serine, and X⁴ being threonine;
(A5) X¹ being alanine and X⁴ being alanine; preferably X¹ being alanine, X² being serine, and X⁴ being alanine;
(A6) X¹ being glycine and X⁴ being alanine; preferably X¹ being glycine, X² being serine, and X⁴ being alanine;
(A7) X¹ being phenylalanine and X⁴ being threonine; preferably X¹ being phenylalanine, X² being serine, and X⁴ being threonine; and
(A8) X¹ being phenylalanine and X⁴ being serine; preferably X¹ being phenylalanine, X² being serine, and X⁴ being serine.

Among these embodiments (A1) to (A8), the embodiment (A1) is preferable from the viewpoint that the phosphorylation of glycerol is performed with higher efficiency.

A more specific embodiment of the phosphorylating enzyme which can be used in the method of the present invention is an enzyme in which X¹, X², and X⁴ in the sequence (1) are any of (B1) to (B11) below:

(B1) X¹ being glycine, X² being serine, X³ being tyrosine, and X⁴ being threonine;
(B2) X¹ being glycine, X² being alanine, X³ being tyrosine, and X⁴ being threonine;
(B3) X¹ being glycine, X² being serine, X³ being tyrosine, and X⁴ being serine;
(B4) X¹ being glycine, X² being aspartic acid, X³ being tyrosine, and X⁴ being threonine;
(B5) X¹ being alanine, X² being serine, X³ being tyrosine, and X⁴ being serine;
(B6) X¹ being alanine, X² being serine, X³ being tyrosine, and X⁴ being threonine;
(B7) X¹ being alanine, X² being serine, X³ being tyrosine, and X⁴ being alanine;
(B8) X¹ being glycine, X² being serine, X³ being tyrosine, and X⁴ being alanine;
(B9) X¹ being phenylalanine, X² being serine, X³ being tyrosine, and X⁴ being threonine;
(B10) X¹ being phenylalanine, X² being serine, X³ being tyrosine, and X⁴ being serine; and
(B11) X¹ being glycine, X² being serine, X³ being tryptophan, and X⁴ being serine.

Among these embodiments (B1) to (B11), the embodiment (B1) is preferable from the viewpoint that the phosphorylation of glycerol is performed with higher efficiency.

In the amino acid sequence of a class A acid phosphatase, the position where the active center is positioned is known; thus those skilled in the art would be able to determine where to place the active center represented by the sequence (1), as appropriate in the amino acid sequence of a phosphorylating enzyme to be used in the present invention.

Additionally, in the amino acid sequence of a class A acid phosphatase, it is known that two active centers are present; the active center represented by the sequence (1) is located on the N-terminal side of these two active centers. The other of these two active centers, which is the active center located on the C-terminal side, usually has an amino acid sequence of RX₅HX₂S wherein each X independently denotes any amino acid, and is joined to the active center represented by sequence (1), usually via 20 to 50 amino acids, preferably via 30 to 40 amino acids, from the C-terminal end of the active center represented by the sequence (1).

Also in the amino acid sequence of a class A acid phosphatase, the amino acid sequences on both the N-terminal and C-terminal sides of the active center represented by the sequence (1) are known; thus those skilled in the art would be able to determine the amino acid sequence located outside the active center represented by the sequence (1), as appropriate in the amino acid sequence of a phosphorylating enzyme to be used in the present invention.

The position of the active center represented by the sequence (1) in the entire amino acid sequence of a phosphorylating enzyme according to embodiment (α) is not limited in particular, as long as the enzyme has an activity to catalyze the phosphorylation of glycerol. For example, the active center represented by the sequence (1) is placed at such a position that the histidine (His) residue at the 7-th position in the amino acid sequence represented by the sequence (1) is located between positions 120 and 160, preferably between positions 125 and 155, further preferably between positions 130 and 150, from the N-terminus of the polypeptide employed as the phosphorylating enzyme (class A acid phosphatase). In addition, the phosphorylating enzyme which is used in the method for producing a phosphorylated glycerol according to the present invention may have, besides the active center represented by the sequence (1), a histidine residue as a second active center at such a position that the histidine residue is located between positions 160 and 200, preferably between positions 165 and 195, further preferably between positions 170 and 190, from the N-terminus of the polypeptide employed as the phosphorylating enzyme.

One embodiment of the phosphorylating enzyme which can be used in the present invention further includes a polypeptide as defined in (I) or (II) below:

(I) a polypeptide that has the amino acid sequence set forth in SEQ ID NO: 2, on the N-terminal side of a stretch of the amino acid sequence coding for the active center represented by the sequence (1), or
(II) a polypeptide that comprises, on the N-terminal side of a stretch of the amino acid sequence coding for the active center represented by the sequence (1), an amino acid sequence having one or a few amino acids substituted, deleted, inserted, or added in the amino acid sequence set forth in SEQ ID NO: 2 and has an activity to catalyze the glycerol phosphorylating reaction which is comparable or superior to that of the polypeptide as defined in (I).

The amino acid sequence set forth in SEQ ID NO: 2 specifically is: PDERLVLAPPPAPGSAAQ (SEQ ID NO: 2)

The amino acid sequence set forth in SEQ ID NO: 2 may be joined directly to a stretch of the amino acid sequence coding for the active center represented by the sequence (1). Alternatively, the amino acid sequence set forth in SEQ ID NO: 2 may be joined to a stretch of the amino acid sequence coding for the active center represented by the sequence (1), for example, via 70 to 130 amino acids, preferably via 80 to 120 amino acids, further preferably via 90 to 110 amino acids, as long as the activity of the enzyme to catalyze the glycerol phosphorylating reaction is not disturbed. In addition, the phosphorylating enzyme according to embodiment (α) may comprise any amino acid sequence, for example, of 5 to 50 amino acids, preferably of 8 to 40 amino acids, further preferably of 10 to 30 amino acids, on the N-terminal side of the amino acid sequence set forth in SEQ ID NO: 2, as long as the activity of the enzyme to catalyze the glycerol phosphorylating reaction is not disturbed. Further, the phosphorylating enzyme according to embodiment (α) may comprise any amino acid sequence, for example, of 60 to 120 amino acids, preferably of 70 to 110 amino acids, further preferably of 80 to 100 amino acids, on the C-terminal side of a stretch of the amino acid sequence coding for the active center represented by the sequence (1), as long as the activity of the enzyme to catalyze the glycerol phosphorylating reaction is not disturbed.

In the amino acid sequence in which a phosphorylating enzyme according to embodiment (α) is composed, the position of the amino acid sequence set forth in SEQ ID NO: 2 is not limited in particular, as long as the enzyme has an activity to catalyze the phosphorylation of glycerol. For example, the amino acid sequence set forth in SEQ ID NO: 2 is placed at such a position that the first amino acid residue in the amino acid sequence set forth in SEQ ID NO: 2 is located between positions 5 and 40, preferably between positions 8 and 35, further preferably between positions 10 and 30, from the N-terminus of the polypeptide employed as the phosphorylating enzyme according to the embodiment (α).

In addition, in a polypeptide as defined in (II) above, the number of amino acids that are substituted, deleted, inserted, or added in the amino acid sequence set forth in SEQ ID NO: 2 is not limited in particular, with the proviso that the polypeptide has an activity to catalyze the glycerol phosphorylating reaction which is comparable or superior to that of the corresponding parent polypeptide. For example, the amino acid sequence set forth in SEQ ID NO: 2 has one or a few amino acids, or, for example, 1 to 10 amino acids, more preferably 1 to 8 amino acids, further preferably 1 to 5 amino acids, particularly preferably 1 to 3 amino acids, or 1 or 2 amino acids substituted, deleted, inserted, or added therein. Methods for obtaining mutants that have an amino acid(s) substituted, deleted, inserted, or added will be described in detail below in the section entitled “2. Preparation of phosphorylating enzymes.”

In cases of adding an amino acid to a given amino acid sequence, the number of amino acids to be added is, for example, from 1 to 10, more preferably from 1 to 8, further preferably from 1 to 5, particularly preferably from 1 to 3, or 1 or 2. Furthermore, in addition to the added amino acid(s), a sequence for protein purification, such as His tag, may optionally added at the C-terminus of the modified amino acid sequences.

In cases of deleting an amino acid from a given amino acid sequence, the number of amino acids to be deleted is, for example, from 1 to 10, more preferably from 1 to 8, further preferably from 1 to 5, particularly preferably from 1 to 3, or 1 or 2.

In cases of inserting an amino acid into a given amino acid sequence, the number of amino acids to be inserted is, for example, from 1 to 10, more preferably from 1 to 8, further preferably from 1 to 5, particularly preferably from 1 to 3, or 1 or 2.

In cases of substituting an amino acid in a given amino acid, a conservative substitution can be made based on properties of the side-chain functional groups of amino acids. In addition, a non-conservative substitution may be made in which the property of an amino acid residue before the substitution is different from that of an amino acid residue after the substitution, as long as the resulting polypeptide has an activity to catalyze the glycerol phosphorylating reaction which is comparable or superior to that of the corresponding parent polypeptide. Natural amino acids are classified, based on their side-chain functional groups, into categories of non-polar amino acids, of non-charged amino acids, of acidic amino acids, and of basic amino acids. A conservative substitution refers to a substitution having an amino acid residue that belongs to the same category into which the parent amino acid residue and the substituted amino acid residue are classified. Here, a “non-polar amino acid” specifically includes alanine, valine, leucine, isoleucine, proline, methionine, phenylalanine, and tryptophan; a “non-charged amino acid” specifically includes glycine, serine, threonine, cysteine, tyrosine, asparagine, and glutamine; an “acidic amino acid” specifically includes aspartic acid and glutamic acid; and a “basic amino acid” specifically includes lysine, arginine, and histidine.

Further, another embodiment of the phosphorylating enzyme which can be used in the present invention includes any of the polypeptides as defined in (III) to (VI) below:

(III) a polypeptide that has the amino acid sequence set forth in any of SEQ ID NOs: 26 to 32 and 106 to 110, on the N-terminal side of the amino acid sequence represented by the sequence (1);
(IV) a polypeptide that has, on the N-terminal side of the amino acid sequence represented by the sequence (1), an amino acid sequence having one or several amino acids substituted, deleted, inserted, or added in the amino acid sequence set forth in any of SEQ ID NOs: 26 to 32 and 106 to 110 and has an activity to catalyze the glycerol phosphorylating reaction which is comparable or superior to that of its corresponding polypeptide as defined in (III);
(V) a polypeptide that has the amino acid sequence set forth in any of SEQ ID NOs: 33 to 39 and 111 to 115, on the C-terminal side of the amino acid sequence represented by the sequence (1); and
(VI) a polypeptide that has, on the C-terminal side of the amino acid sequence represented by the sequence (1), an amino acid sequence having one or several amino acids substituted, deleted, inserted, or added in the amino acid sequence set forth in any of SEQ ID NOs: 33 to 39 and 111 to 115 and has an activity to catalyze the glycerol phosphorylating reaction which is comparable or superior to that of its corresponding polypeptide as defined in (V).

SEQ ID NOs: 26 to 32, 106, and 107 correspond to the amino acid sequences on the N-terminal side of the active center represented by sequence (1), in SEQ ID NOs: 3 to 9, 104, and 105, respectively. The specific amino acid sequences set forth in SEQ ID NOs: 26 to 32, 106, and 107 are as indicated below.

SEQ ID NO: 26:
MDAGYLTPATQPDATQYLPPPPQAGSARQAADDHAFESTRGLKG
GARWALATSDADLRIEALLRSFSCAAGFTIDASKAPRLAALIHR
MDVSEIPDMRNAKASWHRARPFVGNTQSICTEDDRSHLATS
SEQ ID NO: 27:
MDAHGYLEKSELPDSLQLVPPPPQDDSAALANDETVSKAMLALR
GTPRWELAAQDAVLRFPAAATHFSCALGIQIDQTSTPHLVRVLE
RSMRDASTATSAAKARYQRPRPFMRNAQPMCTPDDDAALRKN
SEQ ID NO: 28:
MDHPHGYLTAENTPNAANFLPPPPAEGSLREQADIAAYRAMRSL
EGSERWAIARADNEIETPGAPRAFDCALGFKFEPEQMPTLTLLM
GKMLGDLEMIQTPAKKGYFRKRPFVVEPLPTCIAPETWLAAS
SEQ ID NO: 29:
MDTAPYLAAGQYPDGMAILPPPPALDSPGAALDMAVFRATRKLE
GTPRWRIATDDVTNDPLRRNACAMGMVLDVKTAPALARLLDRAG
TGPVVGRVKAAYQVPRPYLREDGPICEAKTAHLASN
SEQ ID NO: 30:
MDLSQSVSAHTEKSEPSSTYHFHSDPLLYLAPPPTSGSPLQAHD
DQTFNSTRQLKGSTRWALATQDADLHLASVLKDYACAAGMNLDI
AQLPHLANLIKRALRTEYDDIGRAKNNWNRKRPFVDTDQPICTE
KDREGLGKQ
SEQ ID NO: 31:
MDIGINSDPQLAWSETQFVSPQQVDLARLLPPPPAMDSAEQRDE
IALLLQLQKDRTPDMVAFAQADAAREVFRFTDVVGPQFTAEKLP
VAAAFFKAVKENGDAILGNAKKHWDRPRPYAASSQIDPCVPKPG
N
SEQ ID NO: 32:
MDTSATAQGGILPDSAAPDERLVLAPPPAPGSAAQSDDDRVFHV
TRALKDTPRWKLAQSDADLDPAHVVRDFSCAAGFEIDLARAPHL
ARVLERIRHAVGHRTSDVKKYWHRTRPFVGTNLPICTSPEGLGL
N
SEQ ID NO: 106:
MDTGPTVTDPHFKLAPGYLEPASLPVRLALLGGPPKPDSAAFAR
DEEARRAALALRGSAREKLAATDAELTFPAPAKSFSCALGTDIN
EKKTPHLYAMMQHVLTDAGGSTYAGKNAYNRTRPFVQHDEGTCR
KDMEPVLRTD
SEQ ID NO: 107:
MEEAKPFITSQELDLTQYLPAPPADDSAQTQAELKELLQIQATR
TPEQEKAAIADAQENVWRFADVMGPGFDAEKLPKTAALFERIVA
TEDVVDDHAKKAFNRPRPYMLDEQIHPLLKKSKS

SEQ ID NOs: 108 to 110 correspond to the amino acid sequences on the N-terminal side of the active center, in SEQ ID NOs: 10 to 12 representing the amino acid sequences of class A acid phosphatases. The specific amino acid sequences set forth in SEQ ID NOs: 108 to 110 are as indicated below.

SEQ ID NO: 108:
MDALLDGYLSEAEMPDSLLLLSPPPEHNSSLFDLDLEHAKKAVE
SKDKERFLQAARDADLSFPFAVKSFEPILGIEISETKTPKFYVL
MRRVMTDAGLSTYAAKNHYKRERPFMVNNQKTCTPDQENILYKV
SEQ ID NO: 109:
MDSAPSLEKDKLAAAAPKGYLSEEATPNLVAILPPPPAGHSAAE
AADRAVYNAARAFQGSPRWALATDDVADGGAALLQDYACVLGQR
IDQASVPDLMRLLDRARIDIARATRVAKRRYRRLRPFVGNDLPI
CVARTAELADS
SEQ ID NO: 110:
MDSSLFGYTAQAQQFTLPDGRAFLPPPPQAEEPAQQADLRAFEK
TRGLKDKARWKLAQNDANLNPSHVIKDFSCAAGFNLDPEKLPAM
VNLLTSLAQPVEQDVSNEKDFWKRRRPFVGTNKDICTAHSDGLD
NS

SEQ ID NOs: 33 to 39, 111, and 112 correspond to the amino acid sequences on the C-terminal side of the active center represented by sequence (1), in SEQ ID NOs: 3 to 9, 104, and 105, respectively. The specific amino acid sequences set forth in SEQ ID NOs: 33 to 39, 111, and 112 are as indicated below.

SEQ ID NO: 33:
LLGWSTALVLAELLPDRSTEILQRGRVFGESRIVCGVHWASDVL
EGYMTGAGDIAAMHGNPAFRADLDAARTELEGLRHEAPKPNPQA
CTIEHDAAAHSPL
SEQ ID NO: 34:
AIGWTWGLILSEIAPAHRDALLARGRAFGDSRLVCNVHWQSDVI
QGRMVGAAAVAALHGNPAFEKDLAAARREIEKAQAKQPTAAAAA
ACNAEREALKTVLPGVM
SEQ ID NO: 35:
ALGWAWGLVLAELAPDRADAILRRGLAYGESRAVCGVHYPSDVE
AGRIVGATIVTRLKADPAFQADFAKAKEEFDAARAAATEATAAC
PASLARQ
SEQ ID NO: 36:
ANGWLEAQILAEVMPDKATAILARGRAYGESRAICGSHSKSAVE
AGYMAGASVFAVLQTSPAYQRDLAAARQEAARLRTTAPRPDAQS
CVAEAEALRVRP
SEQ ID NO: 37:
TIGWSVALILAELIPDHAANILQRGQIFGTSRIVCGAHWFSDVQ
AGYIMASGEIAALHGDADFRRDMELARKELEKARTSAHTPDDLL
CKIEQSAR
SEQ ID NO: 38:
TYGTLMGIILANMVPEKAQALAARAEQYRFNREIGGVHYPSDVA
AGRITGTVIAAFLFNSPEFQQQYAAARAEVRSALGLAQ
SEQ ID NO: 39:
TAGYGMALLLAHLMPEHASAILQRGRVFGESRIVCGAHWKSDVQ
AGYLNASSLMDVLLARPELQDDLAAARQELLAMQGTAPVPDAGT
CAVEHDAAIHSLLSE
SEQ ID NO: 111:
SAAGWAWGLVLAEVQPARATELLARGLAFGQSRVVCNAHWQSDV
DAGRIMGAATVAVLHDNPAFLADLAAAKREVQDATNANLKPTED
CAAERVALSLSMH
SEQ ID NO: 112:
STIGYLMATVLGEMVPEKRNALFARASGYAENRLVAGFHYRSDT
VMSRTGAALIAQKMEEQPDFKTEFDAAKAELRAQSGLK

SEQ ID NOs: 113 to 115 correspond to the amino acid sequences on the C-terminal side of the active center, in SEQ ID NOs: 10 to 12 representing the amino acid sequences of class A acid phosphatases. The specific amino acid sequences set forth in SEQ ID NOs: 113 to 115 are as indicated below.

SEQ ID NO: 113:
AVGWAWSLVLIKLFPDKQEEILKRGHDFGESRVICNAHWYSDVE
MGRVMGRAAVECLCVNSAFLSDLEEVKKEMAGA
SEQ ID NO: 114:
SQGWAYGLIMANLMPEKATQFLVRSRLYGESRVVCGVHWLSDIE
AARTGASALVAVLLADPGFRTDLERARTDLKRALSGEGAKPDPA
LCAREDAAARQPLL
SEQ ID NO: 115:
TWGWLTASILASALPDRATQIMQRGRIFGESRIVCGVHWKSDVQ
AGYMNGSAIFAALQEQPTFTEQMAKVRQELLALRDAKTAPDAKT
CAVEQQAAQD

In the present invention, a phosphorylating enzyme according to embodiment (α) may comprise a combination of the sequence (1), one selected from the N-terminal sequences (SEQ ID NOs: 26 to 32 and 106 to 110), and one selected from the C-terminal sequences (SEQ ID NOs: 33 to 39 and 111 to 115), as long as the activity of the enzyme to catalyze the glycerol phosphorylating reaction is not disturbed.

The same as in the above-described polypeptides as defined in (II) applies to polypeptides as defined in (IV) or (VI), for the number of amino acids to be substituted, deleted, inserted, or added, the type of amino acid to be substituted, and the like.

A phosphorylating enzyme according to embodiment (α) which can be used in the method of the present invention includes more specifically one including a polypeptide as defined in (VII) or (VIII) below:

(VII) a polypeptide that has the amino acid sequence set forth in any of SEQ ID NOs: 3 to 9, 13, 104, 105, 116, 117, and 119 to 121, or
(VIII) a polypeptide that includes an amino acid sequence having, in the amino acid sequence set forth in any of SEQ ID NOs: 3 to 9, 13, 104, 105, 116, 117, and 119 to 121, one or several amino acids substituted, deleted, inserted, or added in a region outside the active center including the amino acid sequence represented by the sequence (1) and has an activity to catalyze the glycerol phosphorylating reaction which is comparable or superior to that of its corresponding polypeptide as defined in (VII).

The same as in the above-described polypeptides as defined in (II) applies to polypeptides as defined in (VIII), for the number of amino acids to be substituted, deleted, inserted, or added, the type of amino acid to be substituted, and the like. In addition, when an amino acid sequence in which one of these polypeptides is composed comprises a sequence corresponding to the amino acid sequence set forth in SEQ ID NO: 2, it is preferable that a mutation is introduced into a region outside of such a sequence.

In a method for producing a phosphorylated glycerol according to the present invention, the phosphorylating enzyme may be used alone by selecting one from among the above-described phosphorylating enzymes, or in combination of two or more of them.

(1-2) Phosphorylating Enzymes According to Embodiment (β)

An embodiment of the phosphorylating enzyme according to embodiment (β) which can be used in the method of the present invention includes one including a polypeptide as defined in (i) or (ii) below:

(i) a polypeptide that has the amino acid sequence set forth in SEQ ID NO: 2 and has an activity to catalyze the glycerol phosphorylating reaction, or
(ii) a polypeptide that has an amino acid sequence having one or several amino acids substituted, deleted, inserted, or added in the amino acid sequence set forth in SEQ ID NO: 2 and has an activity to catalyze the glycerol phosphorylating reaction which is comparable or superior to that of the polypeptide as defined in (i).

The same as in the above-described polypeptides as defined in (II) applies to polypeptides as defined in (ii) above, for the number of amino acids to be substituted, deleted, inserted, or added, the type of amino acid to be substituted, and the like.

The amino acid sequence set forth in SEQ ID NO: 2, and amino acid sequences having one or several amino acids substituted, deleted, inserted, or added in the amino acid sequence set forth in SEQ ID NO: 2 are a partial amino acid sequence that is located on the N-terminal side of the active center in the amino acid sequence of the phosphorylating enzyme. In the amino acid sequence of a phosphorylating enzyme according to embodiment (β), it is suitable that the amino acid sequence set forth in SEQ ID NO: 2 is located on the N-terminal side of the active center, including, in particular, embodiments in which the first amino acid residue in the amino acid sequence set forth in SEQ ID NO: 2 is located between positions 5 and 40, preferably between positions 8 and 35, further preferably between positions 10 and 30, from the N-terminus of the amino acid sequence of the phosphorylating enzyme.

In polypeptides as defined in (i) and (ii) above, the amino acid sequence of the active center is any amino acid sequence that is not limited in particular, with the proviso that it can serve as the active center of a class A acid phosphatase, and preferably is the amino acid sequence represented by the sequence (1) as described above. The amino acid sequence represented by the sequence (1) is as described above in the section entitled “(1-1) Phosphorylating enzymes according to embodiment (α).”

In polypeptides as defined in (i) and (ii) above, wherein the polypeptide comprises as the active center the amino acid sequence represented by the sequence (1), the amino acid sequence set forth in SEQ ID NO: 2 and a stretch of the amino acid sequence coding for the active center represented by the sequence (1) may be joined directly. Alternatively, they may be joined, for example, via 70 to 130 amino acids, preferably via 80 to 110 amino acids, further preferably via 90 to 110 amino acids, as long as the activity of the polypeptide to catalyze the glycerol phosphorylating reaction is not disturbed. In addition, polypeptides as defined in (i) and (ii) above may have any amino acid sequence, for example, of 5 to 50 amino acids, preferably of 8 to 40 amino acids, further preferably of 10 to 30 amino acids, on the N-terminal side of the amino acid sequence set forth in SEQ ID NO: 2, as long as the activity of the polypeptide to catalyze the glycerol phosphorylating reaction is not disturbed.

Further, polypeptides as defined in (i) and (ii) above may have any amino acid sequence, for example, of 60 to 120 amino acids, preferably of 70 to 110 amino acids, further preferably of 80 to 100 amino acids, on the C-terminal side of a stretch of the amino acid sequence coding for the active center, as long as the activity of the polypeptide to catalyze the glycerol phosphorylating reaction is not disturbed. In polypeptides as defined in (i) and (ii) above, the amino acid sequence on the C-terminal side of the active center may be similar to that on the C-terminal side of the active center of a class A acid phosphatase.

A phosphorylating enzyme according to embodiment (β) may further comprise at least one of the amino acid sequences as defined in (iii) to (vi) below:

(iii) a polypeptide that has, on the N-terminal side of the amino acid sequence set forth in SEQ ID NO: 2, the amino acid sequence set forth in SEQ ID NO: 40 and has an activity to catalyze the glycerol phosphorylating reaction;
(iv) a polypeptide that has, on the N-terminal side of the amino acid sequence set forth in SEQ ID NO: 2, an amino acid sequence having one or a few amino acids substituted, deleted, inserted, or added in the amino acid sequence set forth in SEQ ID NO: 40 and has an activity to catalyze the glycerol phosphorylating reaction which is comparable or superior to that of the polypeptide as defined in (iii);
(v) a polypeptide that has, on the C-terminal side of the amino acid sequence set forth in SEQ ID NO: 2, the amino acid sequence set forth in SEQ ID NO: 41 and has an activity to catalyze the glycerol phosphorylating reaction; and
(vi) a polypeptide that has, on the C-terminal side of the amino acid sequence set forth in SEQ ID NO: 2, an amino acid sequence having one or several amino acids substituted, deleted, inserted, or added in the amino acid sequence set forth in SEQ ID NO: 41 and has an activity to catalyze the glycerol phosphorylating reaction which is comparable or superior to that of the polypeptide as defined in (v).

SEQ ID NO: 40 corresponds to the amino acid sequence in SEQ ID NO: 9 that is located on the N-terminal side of the amino acid sequence corresponding to SEQ ID NO: 2. SEQ ID NO: 41 corresponds to the amino acid sequence in SEQ ID NO: 9 that is located on the C-terminal side of the amino acid sequence corresponding to SEQ ID NO: 2. The specific amino acid sequences of these are as indicated below.

SEQ ID NO: 40:
MDTSATAQGGILPDSAA
SEQ ID NO: 41:
SDDDRVFHVTRALKDTPRWKLAQSDADLDPAHVVRDFSCAAGFE
IDLARAPHLARVLERIRHAVGHRTSDVKKYWHRTRPFVGTNLPI
CTSPEGLGLNASYPSGHTTAGYGMALLLAHLMPEHASAILQRGR
VFGESRIVCGAHWKSDVQAGYLNASSLMDVLLARPELQDDLAAA
RQELLAMQGTAPVPDAGTCAVEHDAAIHSLLSE

The same as in the above-described polypeptides as defined in (II) applies to polypeptides as defined in (iv) or (vi) above, for the number of amino acids to be substituted, deleted, inserted, or added, the type of amino acid to be substituted, and the like.

A phosphorylating enzyme according to embodiment (β) which can be used in the method of the present invention includes more specifically one including a polypeptide as defined in (vii) or (viii) below:

(vii) a polypeptide that has the amino acid sequence set forth in any of SEQ ID NOs: 9, 15 to 17, 19, 22, and 25; or
(viii) a polypeptide that includes an amino acid sequence having, in the amino acid sequence set forth in any of SEQ ID NOs: 9, 15 to 17, 19, 22, and 25, one or several amino acids substituted, deleted, inserted, or added in a region outside the amino acid sequence set forth in SEQ ID NO: 2 and has an activity to catalyze the glycerol phosphorylating reaction which is comparable or superior to that of the polypeptide as defined in (vii).

The same as in the above-described polypeptides as defined in (II) applies to polypeptides as defined in (iv) or (vi) above, for the number of amino acids to be substituted, deleted, inserted, or added, the type of amino acid to be substituted, and the like.

In a method for producing a phosphorylated glycerol according to the present invention, the phosphorylating enzyme may be used alone by selecting one phosphorylating enzyme from among phosphorylating enzymes of one type, or alternatively in combination of two or more phosphorylating enzymes. In addition, a phosphorylating enzyme according to embodiment (α) and a phosphorylating enzyme according to embodiment (β) may be used in combination.

### 2. Preparation of Phosphorylating Enzymes

An enzyme as described above having a catalytic activity in a glycerol phosphorylating reaction can be obtained, for example, from a culture of a microorganism that possesses such an enzyme. Alternatively, an enzyme as described above having a catalytic activity in a glycerol phosphorylating reaction can be prepared as a recombinant protein in accordance with conventionally known methods, by obtaining a base sequence encoding such a phosphorylating enzyme of interest from a microorganism possessing the enzyme.

A phosphorylating enzyme including one of the amino acid sequences set forth in SEQ ID NOs: 3 to 9, 104, and 105 can be obtained from particular microorganisms as described below, respectively.

Accordingly, a phosphorylating enzyme including the amino acid sequence set forth in SEQ ID NO: 3 is available from Gluconacetobacter hansenii. A phosphorylating enzyme including the amino acid sequence set forth in SEQ ID NO: 4 is available from Xanthomonas oryzae; a phosphorylating enzyme including the amino acid sequence set forth in SEQ ID NO: 5 is available from Brevundimonas diminuta; a phosphorylating enzyme including the amino acid sequence set forth in SEQ ID NO: 6 is available from Sphingomonas trueperi; a phosphorylating enzyme including the amino acid sequence set forth in SEQ ID NO: 7 is available from Zymomonas mobilis; a phosphorylating enzyme including the amino acid sequence set forth in SEQ ID NO: 8 is available from Desulfovibrio magneticus; a phosphorylating enzyme including the amino acid sequence set forth in SEQ ID NO: 9 is available from Gluconobacter oxydans. A phosphorylating enzyme including the amino acid sequence set forth in SEQ ID NO: 104 is available from Serratia plymutica. A phosphorylating enzyme including the amino acid sequence set forth in SEQ ID NO: 105 is available from Rahnella aquatilis.

A phosphorylating enzyme including one of the amino acid sequences set forth in SEQ ID NOs: 3 to 9, 104, and 105 can be obtained by culturing the corresponding microorganism and treating cultured cells. To this end, more specifically, the methods described below are illustrated.

In cases when a phosphorylating enzyme including the amino acid sequence set forth in SEQ ID NO: 3 is obtained from cultured cells of Gluconacetobacter hansenii, culture conditions can be set as appropriate from culture methods known for this microorganism, for example, including aerobic conditions at a culture temperature of 30° C. in a medium containing 1% polypeptone, 0.2% dry yeast extract, and 0.1% magnesium sulfate.

In cases when a phosphorylating enzyme including the amino acid sequence set forth in SEQ ID NO: 4 is obtained from cultured cells of Xanthomonas oryzae, culture conditions can be set as appropriate from culture methods known for this microorganism, for example, including aerobic conditions at a culture temperature of 30° C. in a medium containing 1% polypeptone, 0.2% dry yeast extract, and 0.1% magnesium sulfate.

In cases when a phosphorylating enzyme including the amino acid sequence set forth in SEQ ID NO: 5 is obtained from cultured cells of Brevundimonas diminuta, culture conditions can be set as appropriate from culture methods known for this microorganism, for example, including aerobic conditions at a culture temperature of 30° C. in a medium containing 1% polypeptone, 0.2% dry yeast extract, and 0.1% magnesium sulfate.

In cases when a phosphorylating enzyme including the amino acid sequence set forth in SEQ ID NO: 6 is obtained from cultured cells of Sphingomonas trueperi, culture conditions can be set as appropriate from culture methods known for this microorganism, for example, including aerobic conditions at a culture temperature of 26° C. in a medium containing 5% peptone and 3% beef extract.

In cases when a phosphorylating enzyme including the amino acid sequence set forth in SEQ ID NO: 7 is obtained from cultured cells of Zymomonas mobilis, culture conditions can be set as appropriate from culture methods known for this microorganism, for example, including aerobic conditions at a culture temperature of 30° C. in a medium containing 0.5% dry yeast extract and 2% glucose.

In cases when a phosphorylating enzyme including the amino acid sequence set forth in SEQ ID NO: 8 is obtained from cultured cells of Desulfovibrio magneticus, culture conditions can be set as appropriate from culture methods known for this microorganism, for example, including anaerobic conditions at a culture temperature of 30° C. in a medium containing 0.02% potassium dihydrogenphosphate, 60 ppm ammonium chloride, 50 ppm cysteine, 0.058% sodium fumarate, 0.044% sodium pyruvate, 0.2% iron quinate solution, 0.4% Wolfe's vitamin solution, and 0.2% Wolfe's mineral solution.

In cases when a phosphorylating enzyme including the amino acid sequence set forth in SEQ ID NO: 9 is obtained from cultured cells of Gluconobacter oxydans, culture conditions can be set as appropriate from culture methods known for this microorganism, for example, including aerobic conditions at a culture temperature of 30° C. in a medium containing 0.5% polypeptone, 0.5% dry yeast extract, 0.5% glucose, and 0.1% magnesium sulfate.

In cases when a phosphorylating enzyme including the amino acid sequence set forth in SEQ ID NO: 104 is obtained from cultured cells of Serratia plymutica, culture conditions can be set as appropriate from culture methods known for this microorganism, for example, including aerobic conditions at a culture temperature of 37° C. in a medium containing 1% polypeptone, 0.2% dry yeast extract, and 0.1% magnesium sulfate.

In cases when a phosphorylating enzyme including the amino acid sequence set forth in SEQ ID NO: 105 is obtained from cultured cells of Rahnella aquatilis, culture conditions can be set as appropriate from culture methods known for this microorganism, for example, including aerobic conditions at a culture temperature of 30° C. in a medium containing 1% polypeptone, 0.2% dry yeast extract, and 0.1% magnesium sulfate.

After the culturing is completed, usual methods for enzyme isolation can be employed to collect the phosphorylating enzyme from the culture. Methods for enzyme isolation include, for example, those in which cultured cells are disrupted by sonication to prepare a cell extract solution, followed by known methods for enzyme purification, such as ammonium sulfate fractionation, ion exchange, chromatofocusing, gel filtration, and the like. In addition, a cultured broth containing an enzyme secreted outside the cells may be used as an enzyme solution.

Also, a phosphorylating enzyme including one of the amino acid sequences set forth in SEQ ID NOs: 3 to 9, 104, and 105 can be prepared as a recombinant protein, which then can be used in the method of the present invention. In cases when the phosphorylating enzyme is obtained as a recombinant protein, the preparation of the recombinant protein can be performed based on known genetic engineering procedures. In usual cases, a phosphorylating enzyme can be produced as a recombinant protein by inserting a polynucleotide (DNA fragment) encoding the amino acid sequence of a given phosphorylating enzyme into a vector, introducing the vector into a host cell to obtain a transformant, and culturing the transformant. The polynucleotide sequences that encode phosphorylating enzymes including the amino acid sequences set forth in SEQ ID NOs: 3 to 9, 104, and 105 are represented by SEQ ID NOs: 94 to 100, 122, and 123, respectively. In such cases, a sequence derived from the vector, which is described below, may be incorporated into the polynucleotide, with the proviso that the sequence does not disturb the activity of the phosphorylating enzyme. In addition, the polynucleotide encoding the phosphorylating enzyme may be optimized so that the codon usage is adapted to that of the host cell to be used.

As a vector, use can be made of any vector that allows the expression of the gene encoded by the DNA fragment in an appropriate host cell. Such a vector includes, for example, a plasmid vector, a phage vector, a cosmid vector, a shuttle vector, and the like.

In the present invention, a vector may comprise a regulatory element(s) that is/are operably linked to a polynucleotide (DNA fragment) encoding an enzyme mutant of the present invention as described above, for example, a promoter, for example, a functional promoter such as a T7 promoter, a lacUV5 promoter, a trp promoter, a trc promoter, a tac promoter, an lpp promoter, a tufB promoter, a recA promoter, and a pL promoter; a transcriptional element, for example, an enhancer, a CCAAT box, a TATA box, and an SPI site; a signal sequence, particularly a nuclear localization signal, an endoplasmic reticulum localization signal, PTS1 (Peroxisomal targeting signal 1), and PTS2; and others. Here, by operably linked, it is meant that various regulatory elements such as a promoter and an enhancer that regulate the expression of a gene, and a given gene are linked in a state where they are allowed to operate within the host cell.

A vector to be used in the present invention can be selected as appropriate from among vectors commonly used in the art and can be used. Examples of such a vector includes, more specifically, pET22b(+), pET39b(+), and others.

Host cells are not limited in particular, as long as they can be transformed with an expression vector comprising a polynucleotide (DNA fragment) encoding an above-mentioned phosphorylating enzyme and allow the expression of the phosphorylating enzyme encoded by the polynucleotide.

Examples of such host cells include, more specifically, microorganisms, for example, bacteria of the genera Escherichia, Bacillus, Pseudomonas, Serratia, Brevibacterium, Corynebacterium, Streptococcus, Lactobacillus and the like; actinomycetes of the genera Rhodococcus, Streptomyces, and the like; yeasts of the genera Saccharomyces, Kluyveromyces, Schizosaccharomyces, Zygosaccharomyces, Yarrowia, Trichosporon, Rhodosporidium, Pichia, Candida, and the like; fungi of the genera Neurospora, Aspergillus, Cephalosporium, Trichoderma, and the like; and the others. Among these, bacteria are preferred from the viewpoint of the efficiency of introduction of genes into host cells and of gene expression. Cells of Escherichia coli, a bacterium of the genus Escherichia, are preferable.

Methods for transformation are known and can be selected as appropriate, depending on the type of host cell to be transformed and others. Examples of methods for transformation specifically include methods using competent cells, electroporation, heat shock, or the like.

Transformants can be cultured, based on conventionally known culture conditions, depending on the type of cell used as host cell. In cases when Escherichia coli cells are used as host cell, for example, culture conditions can be a culture temperature of 20 to 40° C., a culture period of 6 to 24 hours, and a pH of the culture medium of 5 to 8 under aerobic conditions, for example, by shaking culture or aeration culture.

A culture medium that is to be used in the present invention is not limited in particular, as long as it allows the host cell to grow and to produce an enzyme mutant of the present invention. Use can be made of media usually employed in the art which contain required amounts of a carbon source, a nitrogen source, inorganic substances, amino acids, nucleic acids, vitamins, and others. In addition, an antibiotic such as ampicillin or tetracycline may be added to the medium as necessary, during the culturing period.

Methods for collecting the phosphorylating enzyme of interest after the culturing is completed may follow the procedures for collecting the phosphorylating enzyme from cultured cells as previously described.

Introduction of a mutation (substitution, deletion, insertion, and/or addition) into a given amino acid sequence can be carried out according to conventionally known methods. Methods for introducing an amino acid mutation include, for example, site-directed mutagenesis, which can be performed by using Inverse PCR-based procedures or commercially available kits, such as QuikChange II Kit (manufactured by Stratagene). These approaches make it possible to obtain a polynucleotide encoding a phosphorylating enzyme having a desired mutation, based on the base sequences set forth in SEQ ID NOs: 94 to 100, 122, and 123. A phosphorylating enzyme mutant can be prepared as a recombinant protein by using the resulting polynucleotide and following known genetic engineering procedures similar to the above.

A phosphorylating enzyme that includes a polypeptide having a mutation as described above includes one in which the amino acid sequence identity of such a polypeptide to the corresponding polypeptide before the mutation is introduced is 20% or higher, preferably 30% or higher, further preferably 40% or higher and which is capable of catalyzing the glycerol phosphorylating reaction at a level comparable or superior to that of the phosphorylating enzyme including the corresponding polypeptide before the mutation is introduced.

The sequence identity between two polypeptides is expressed as a numerical value obtained by optimally aligning the two polypeptides to be compared, dividing the number of positions at which amino acids are identical in both sequences by the total number of the amino acids compared, and multiplying the quotient by 100. Specifically, the sequence identity between two polypeptides can be determined by using a Maximum matching program, “GENETYX Ver. 10” (GENETYX CORPORATION).

### 3. Phosphorylation of Glycerol

In a method for phosphorylating glycerol according to the present invention, the phosphorylation reaction can be carried out by adding glycerol, a phosphate group donor, and an above-described phosphorylating enzyme to a solvent to prepare a reaction composition. Alternatively, the phosphorylation of glycerol may be performed by allowing a culture of an above-described microorganism or transformant that is capable of producing an above-described phosphorylating enzyme to act on glycerol in the presence of a phosphate group donor.

In the method of the present invention, a culture means a cultured broth containing cells, a cultured cell mass, or a treated material thereof. Here, a treated material thereof means, for example, a cell-free extract, a freeze-dried cell preparation, an acetone-dried cell preparation, or a disrupted material of these cell preparations. Such cultures can also be used as an immobilized enzyme or cell preparation Immobilization can be performed using methods well known to those skilled in the art, for example, covalent bonding, physical adsorption, entrapment, and the like.

A phosphate group donor is not limited in particular, as long as it functions as a source of the phosphate for an enzyme mutant of the present invention, whereby a phosphorylated glycerol is yielded, and includes, for example, a polyphosphoric acid represented by formula (1): Hₙ₊₂PₙO₃ₙ₊₁, wherein n≧2. The degree of polymerization of the polyphosphoric acid is not limited in particular, and preferably 2 to 10000 structural units described above are polymerized, further preferably 2 to 1000 structural units described above are polymerized. Examples of a polyphosphoric acid specifically include diphosphoric acid (n=2; pyrophosphoric acid), triphosphoric acid (n=3), tetraphosphoric acid (n=4), and the like. It is also possible that in the formula (1), polyphosphoric acids in which 5 or more phosphoric acid groups are polymerized (n=5 or more) are suitably used as a phosphate group donor. A mixture of two or more of these polyphosphoric acids with different degrees of polymerization may be used as a phosphate group donor.

Further, as a phosphate group donor which can be used in the present invention, use can also be made of esters, salts with alkali metals such as sodium and potassium, salts with alkaline earth metals such as calcium, and ammonium salts of the polyphosphoric acid described above. Examples of esters of polyphosphoric acids specifically include adenosine diphosphate (ADP), adenosine triphosphate (ATP), and others. Examples of salts of polyphosphoric acids specifically include sodium pyrophosphate (Na₄P₂O₇), sodium tripolyphosphate (Na₅P₃O₁₀), potassium tripolyphosphate (K₅P₃O₁₀), sodium tetrapolyphosphate (Na₆P₄O₁₃), sodium hexametaphosphate, ammonium pyrophosphate salt, ammonium tripolyphosphate salt, and others. Moreover, a phosphate group donor includes para-nitrophenyl phosphate, acetyl phosphate, and the like, in addition to the polyphosphoric acid described above.

In the present invention, the phosphate group donor preferably includes polyphosphoric acids, such as diphosphoric acid, triphosphoric acid, and tetraphosphoric acid; esters of polyphosphoric acids, such as adenosine diphosphate (ADP) and adenosine triphosphate (ATP); alkali metal salts of polyphosphoric acids, such as sodium pyrophosphate, sodium tripolyphosphate, potassium tripolyphosphate, sodium tetrapolyphosphate, and sodium hexametaphosphate; para-nitrophenyl phosphate and acetyl phosphate. More preferably, the phosphate group donor includes polyphosphoric acids, and esters and alkali metal salts thereof. Further preferably, the phosphate group donor includes polyphosphoric acids and alkali metal salts thereof, with polyphosphoric acids being preferable from the viewpoint that they are available at low cost. These phosphate group donors may be used alone or in combination of two or more.

Examples of the concentration at which a phosphorylating enzyme is used in the reaction include 0.0001% to 5% by weight, preferably 0.001% to 1% by weight; further preferably 0.001% to 0.5% by weight, in the reaction composition. Examples of the concentration of a phosphate group donor include 0.5% to 20% by weight, preferably 1% to 15% by weight; further preferably 2% to 10% by weight. Examples of the concentration of glycerol include 0.1% to 80% by weight, preferably 1% to 60% by weight; further preferably 5% to 50% by weight.

Examples of the addition ratio of a phosphate group donor per part by weight of a phosphorylating enzyme (polypeptide) include 100 to 30000 parts by weight, preferably 250 to 20000 parts by weight, and further preferably 500 to 15000 parts by weight. Examples of the addition ratio of glycerol per part by weight of a phosphorylating enzyme (polypeptide) include 100 to 100000 parts by weight, preferably 500 to 70000 parts by weight, and further preferably 1000 to 50000 parts by weight.

Examples of the reaction temperature at which the phosphorylation reaction is carried out include 10 to 60° C., preferably 20 to 50° C., further preferably 30 to 40° C.

Examples of the optimal pH of the reaction solution when the phosphorylation reaction is carried out include pH 2 to 7, preferably pH 4 to 6, more preferably pH 3 to 5, further preferably pH 4 to 5. The pH of the reaction solution can be adjusted using hydrochloric acid, citric acid, gluconic acid, succinic acid, acetic acid, tartaric acid, sorbic acid, lactic acid, maleic acid, sulfuric acid, phosphoric acid, malic acid, arginine, aqueous ammonia, diisopropanolamine, diethanolamine, triisopropanolamine, triethanolamine, monoethanolamine, potassium hydroxide, calcium hydroxide, sodium hydroxide, and buffering agents such as salts thereof, and conventionally known buffering agents.

The reaction period when the phosphorylation reaction is carried out is not limited in particular, as long as a phosphorylated glycerol can be produced, and for example, is 1 to 50 hours, preferably 5 to 40 hours, further preferably 10 to 30 hours.

As a reaction solvent, an aqueous solvent, such as ion-exchanged water, distilled water, and ultrapure water can be used in usual cases. In addition, an aqueous solvent may contain an organic solvent such as methanol, trimethylamine, triethylamine, and dimethyl sulfoxide (DMSO), or a non-ionic surfactant such as polyoxyethylene lauryl ether, polyoxyethylene dodecyl ether, polyethylene glycol p-octylphenyl ether (whose trade name is Triton X-100), and polyoxyethylene sorbitan monolaurate (whose trade name is Tween 20), in a range in which advantageous effects of the present invention are not inhibited.

Examples of amounts of an organic solvent added are usually 0.1% to 50% by weight, preferably 1% to 30% by weight, and further preferably 5% to 20% by weight. Examples of amounts of a non-ionic surfactant added are usually 0.0001% to 0.1% by weight, preferably 0.0005% to 0.05% by weight, further preferably 0.001% to 0.01% by weight.

Methods for collecting a phosphorylated glycerol yielded in the above-described reaction can be, for example, methods using various types of column chromatography, such as ion-exchange chromatography, affinity chromatography, adsorption column chromatography, gel filtration chromatography, and reverse phase chromatography, and methods by which the phosphorylated glycerol is precipitated by the addition of an organic solvent such as ethanol or acetonitrile.

In cases when the glycerol phosphorylating reaction is performed under conditions in amounts of 5000 to 50000 parts by weight of glycerol per part by weight of a phosphorylating enzyme (polypeptide) and at a pH of 4 to 6, the phosphorylating enzyme (polypeptide) used includes, for example, a phosphorylating enzyme including one of the amino acid sequences set forth in SEQ ID NOs: 3 to 9, 104, and 105, or a mutant thereof, preferably a phosphorylating enzyme including one of the amino acid sequences set forth in SEQ ID NOs: 4 to 8, or a mutant thereof, further preferably a phosphorylating enzyme including the amino acid sequence set forth in SEQ ID NO: 4, or a mutant thereof.

In a method for producing a phosphorylated glycerol according to the present invention, it is possible to achieve a further higher efficiency of the phosphorylation by adjusting conditions for the phosphorylation reaction and selecting an appropriate phosphorylating enzyme. For example, when the glycerol phosphorylating reaction is performed under conditions in amounts of 5000 to 50000 parts by weight of glycerol per part by weight of a phosphorylating enzyme (polypeptide) and at a pH of 3 to 5, the phosphorylating enzyme (polypeptide) used includes, for example, a phosphorylating enzyme including the amino acid sequence set forth in SEQ ID NO: 3, 4, 8, or 9, or a mutant thereof, further preferably a phosphorylating enzyme including the amino acid sequence set forth in SEQ ID NO: 4, or a mutant thereof.

Further, when the glycerol phosphorylating reaction is performed under conditions in amounts of 2000 to 10000 parts by weight of glycerol per part by weight of a phosphorylating enzyme (polypeptide) and at a pH of 4 to 5, the phosphorylating enzyme (polypeptide) used includes, for example, a phosphorylating enzyme including the amino acid sequence set forth in SEQ ID NO: 3, 5, or 9, or a mutant thereof, further preferably a phosphorylating enzyme including the amino acid sequence set forth in SEQ ID NO: 3, or a mutant thereof.

In the method of the present invention, the hydroxyl group position at which the phosphate group is attached is not limited in particular, as long as glycerol is phosphorylated. A specific example of the phosphorylated glycerol yielded in the method of the present invention is glycerol-3-phosphate (also referred to as glycerophosphate). Glycerophosphate occurs in the a and 13 forms; the glycerophosphate obtained by method of the present invention can include either of these forms. Preferably, the method of the present invention yields glycerophosphate in the a form. In examples described below, it has been found that glycerol-3-phosphate in the a form (also referred to as α-glycerophosphate) is predominantly produced.

### EXAMPLES

The present invention will be more specifically described presenting experimental examples below, which are not intended to limit the present invention thereto.

[Preparation of Phosphorylating Enzymes]

Obtaining of Genes for Phosphorylating Enzymes

Genomic DNA was prepared from each strain of the microorganism indicated below in Table 1 and a gene for a phosphorylating enzyme was amplified by PCR method using primers corresponding to the respective strains of the microorganisms.

TABLE 1

| | SEQ ID NO: | | | | |
|---|---|---|---|---|---|
| SEQ ID NO: | of the base | | | | |
| of the amino | sequence | | | | |
| acid sequence | of a gene | | | | |
| of the | encoding the | | | | |
| phospho- | phospho- | | Primer | | |
| rylating | rylating | | SEQ ID | | |
| enzyme | enzyme | Derived from | NO: | Primer sequence (5′ to 3′) | |
| 3 | 94 | Gluconacetobacter | 42 | FW | TGCACCCATGGACGCAGGGTATCTAACACCTGCGAC |
| | | hansenii | 43 | RV | CAATAAGCTTGAGCGGAGAATGGGCCGCAGCATCATG |
| 4 | 95 | Xanthomonas | 44 | FW | GATCGCCATGGACGCGCATGGATACCTGGAAAAG |
| | | oryzae | 45 | RV | TTCCAAGCTTCATCACACCAGGCAACACCGTCTTC |
| 5 | 96 | Brevundimonas | 46 | FW | AGCTTCCATGGATCATCCGCACGGCTATCTGACCGCC |
| | | diminuta | 47 | RV | ATCAAAGCTTCTGACGCGCCAGCGAGGCGGGGCAGG |
| 6 | 97 | Sphingomonas | 48 | FW | TCAGGCCATGGACACGGCGCCGTACCTCGCCG |
| | | trueperi | 49 | RV | AAGAAAGCTTGGGGCGGACGCGAAGGGCCTCCGC |
| 7 | 98 | Zymomonas | 50 | FW | TCTGGCCATGGATCTTTCTCAAAGCGTTTCAGCT |
| | | mobilis | 51 | RV | TATGAAGCTTGCGAGCGCTTTGTTCAATCTTGC |
| 8 | 99 | Desulfovibrio | 52 | FW | TATGGATCCGCAGCTGGCCTGGTCCGAAACGCAGTTC |
| | | magneticus | 53 | RV | CCTCAAGCTTCTGGGCCAGACCCAGGGCGCTGCGGACTT |
| 9 | 100 | Gluconobacter | 54 | FW | TAACGCCCATGGATACGTCCGCTACCGCCCAAGGCGGC |
| | | oxydans | 55 | RV | AACAAAGCTTTTCGCTGAGCAGGGAATGGATCGCGGC |
| 10 | 101 | Flavobacterium | 56 | FW | CTTAGACCATGGATGCATTGCTGGATGGATATTTG |
| | | johnsoniae | 57 | RV | CCATAAGCTTAGCTCCTGCCATTTCTTTTTTAACCT |
| 11 | 102 | Methylobacterium | 58 | FW | TACGTTCCATGGATTCCGCCCCCTCGCTCGAGAAGGA |
| | | ectorquens | 59 | RV | ATCCAAGCTTGAGCAGGGGCTGGCGCGCGGCCGCAT |
| 12 | 103 | Acetobacter | 60 | FW | CTTAGACCATGGATAGCAGCCTTTTTGGATATACC |
| | | pasteurianus | 61 | RV | AAGTAAGCTTAAAAGCGTCTTGCGCAGCCTGCTGTTC |
| 104 | 122 | Serratia | 124 | FW | GCTGCCACCATGGATACCGGGCCGACGGTAACGGAC |
| | | plymutica | 125 | RV | GATCAAGCTTGTGCATACTCAAAGAGAGTGCAAC |
| 105 | 123 | Rahnella | 126 | FW | GTTCAGGCCATGGAAGAAGCCAAACCCTTTATCACC |
| | | aquatilis | 127 | RV | ACGAAAGCTTTTTCAGGCCAGATTGGGCGCGAAG |

Preparation of Transformants

For SEQ ID NOs: 3 to 7, 9 to 12, 104, and 105, the resultant PCR product was digested with restriction enzymes (NcoI and HindIII), and ligated into a pET22b(+) vector (Novagen) treated with NcoI/HindIII restriction enzymes to obtain a vector for expression of the encoded phosphorylating enzyme. For SEQ ID NO: 8, the resultant PCR product was digested with restriction enzymes (BamHI and HindIII), and ligated into a pET22b(+) vector (Novagen) treated with BamHI/HindIII restriction enzymes to obtain a vector for expression of the encoded phosphorylating enzyme. These vectors further carry, besides a region encoding the phosphorylating enzyme, a T7 promoter, a pelB signal for transport of the expressed protein into the periplasm, a His-tag gene for protein purification, and an ampicillin resistance gene for selection with a drug. The vector obtained by the above-described procedures was transformed into cells of an Escherichia coli (E. coli) BL21(DE3) strain (Novagen) by heat shock method.

Preparation of Phosphorylating Enzymes

Culturing of transformant cells of an E. coli BL21(DE3) strain carrying the vector for expression of the encoded phosphorylating enzyme was performed by incubating them at a culture temperature of 27° C. in an LB medium (manufactured by Nacalai Tesque, Inc. and having a composition of 1% by weight of tryptone, 0.5% by weight of dry yeast extract, 0.5% by weight of sodium chloride) containing 0.2% by weight of glucose, 0.5% by weight of casamino acid, and 0.1 mg/mL of ampicillin. When the optical density of the culture at 600 nm reached 0.5, isopropyl-β-thiogalactopyranoside was added to a concentration of 0.1 mM and additional culturing was performed at a culture temperature of 27° C. for a period of 16 to 24 hours.

After the culture was completed, 15 g of wet cells was suspended in 20 mM Tris-HCl buffer (pH 8.0) containing 150 mM sodium chloride, and then subjected to an ultrasonic homogenizer (TOMY UD201) to obtain a cell extract solution. The cell extract solution was subjected to centrifugation at 20,000×g for 20 minutes to collect the supernatant fraction. To the supernatant, ammonium sulfate was added to a saturation of 60%, followed by centrifugation at 20,000×g for 20 minutes to collect a precipitate. The precipitate after the ammonium sulfate fractionation was suspended in 20 mM Tris-HCl buffer (pH 7.5), and then dialyzed against 20 mM Tris-HCl buffer (pH 7.5) containing 50 mM sodium chloride. After the dialysis was completed, affinity purification was carried out using a His-Trap HP column. Fractions containing the phosphorylating enzyme were collected, and then subjected to dialysis against 20 mM Tris-HCl buffer (pH 7.5) to obtain the phosphorylating enzyme. Conditions for the affinity purification were as follows.

Purification Conditions for Phosphorylating Enzymes

Column: His-Trap HP (manufactured by GE healthcare)
Equilibration buffer: 20 mM Tris-HCl (pH 7.5), 50 mM sodium chloride
Elution buffer: 20 mM Tris-HCl (pH 7.5), 50 mM sodium chloride, 1 M imidazole (elution in a gradient of the elution buffer from 0 to 50%)
Flow rate: 5.0 mL/min
Purification equipment: AKTA prime plus (GE healthcare)

The amino acid sequence and position of the active center of the phosphorylating enzymes obtained are indicated in Table 2.

TABLE 2

| Amino acid | | |
|---|---|---|
| sequence | | |
| of the | Amino acid | |
| phospho- | sequence of | Position of |
| rylating | the active | the active |
| enzyme | center | center |
| SEQ ID | Gly Ala Tyr Pro | Positions 130 to 137 |
| NO: 3 | Ser Gly His Thr | in SEQ ID NO: 3 |
| SEQ ID | Gly Ser Tyr Pro | Positions 131 to 138 |
| NO: 4 | Ser Gly His Thr | in SEQ ID NO: 4 |
| SEQ ID | Gly Ser Tyr Pro | Positions 131 to 138 |
| NO: 5 | Ser Gly His Ser | in SEQ ID NO: 5 |
| SEQ ID | Gly Asp Tyr Pro | Positions 125 to 132 |
| NO: 6 | Ser Gly His Thr | in SEQ ID NO: 6 |
| SEQ ID | Gly Ser Tyr Pro | Positions 142 to 149 |
| NO: 7 | Ser Gly His Thr | in SEQ ID NO: 7 |
| SEQ ID | Ala Ser Tyr Pro | Positions 134 to 141 |
| NO: 8 | Ser Gly His Ser | in SEQ ID NO: 8 |
| SEQ ID | Ala Ser Tyr Pro | Positions 134 to 141 |
| NO: 9 | Ser Gly His Thr | in SEQ ID NO: 9 |
| SEQ ID | Gly Ser Phe Pro | Positions 133 to 140 |
| NO: 10 | Ser Gly His Ala | in SEQ ID NO: 10 |
| SEQ ID | Phe Ser Tyr Pro | Positions 144 to 151 |
| NO: 11 | Ser Gly His Ala | in SEQ ID NO: 11 |
| SEQ ID | Tyr Ala Tyr Pro | Positions 135 to 142 |
| NO: 12 | Ser Gly His Thr | in SEQ ID NO: 12 |
| SEQ ID | Gly Ser Tip Pro | Positions 143 to 150 |
| NO: 104 | Ser Gly His Ser | in SEQ ID NO: 104 |
| SEQ ID | Gly Ser Tip Pro | Positions 123 to 130 |
| NO: 105 | Ser Gly His Ser | in SEQ ID NO: 105 |

### Experimental Example 1

The microorganism-derived phosphorylating enzymes obtained as described above (which are represented by SEQ ID NOs: 3 to 12, 104, and 105) were used to phosphorylate glycerol. Conditions for and results of the glycerol phosphorylating reaction are indicated below.

Phosphorylation of Glycerol

To ultrapure water were added glycerol (4 M or 0.5 M), a polyphosphoric acid as a phosphate group donor (4% by weight; manufactured by Nacalai Tesque, Inc. under a trade name of polyphosphoric acid), and Triton X-100 (0.1% by weight), to prepare a reaction mixture solution. When glycerol was used at a concentration of 4 M, the pH of the solution was adjusted to pH 4.0 or 5.0 with KOH; and when glycerol was used at a concentration of 0.5 M, the pH of the solution was adjusted to pH 4.5 with KOH. Further, the phosphorylating enzyme including one of the amino acid sequences set forth in SEQ ID NOs: 3 to 12, 104, and 105 was added to the reaction mixture solution to make a final concentration of 20 μg/mL. The reaction solution was subjected to reaction in a temperature-controlled air incubator at 37° C. overnight. After that, a 0.1-mL sample of the reaction solution was taken and diluted 50 to 500 times in 50 mM PIPES buffer (pH 7.0). The concentration of glycerol-3-phosphate in the sample was detected by an enzymatic method using glycerol-3-phosphate dehydrogenase under the conditions described below.

Detection Method for Glycerol-3-Phosphate

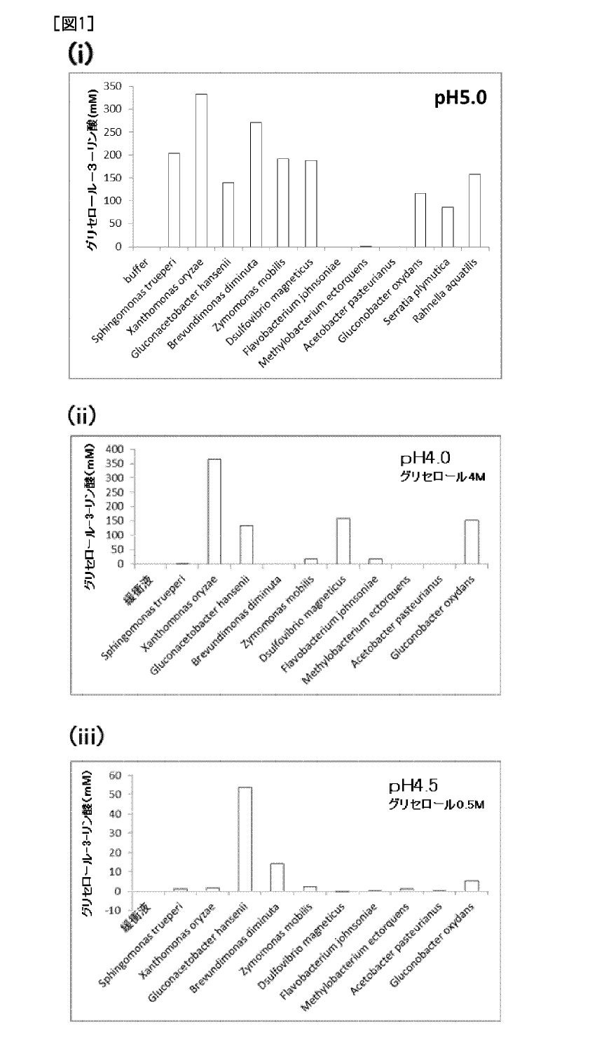

Glycerol-3-phosphate dehydrogenase and potassium ferricyanide were mixed with 50 mM PIPES buffer (pH 7.0) to make final concentrations of 10 U/mL and 2 mM, respectively. To 0.9 mL of this measurement solution was added a 0.1-mL sample of the reaction solution after the phosphorylation reaction, to prepare a reaction mixture, which then was subjected to measurement of the rate of decrease in the absorbance at 420 nm, for 3 minutes at 37° C. A standard curve was prepared from the rates of decrease in the absorbance obtained by using known concentrations of glycerol-3-phosphate, and used to calculate the concentration of glycerol-3-phosphate produced. The results are shown in FIG. 1. In FIG. 1, graph (i) shows the results when the reaction was carried out using 4 M glycerol and at a pH of 5.0, graph (ii) shows the results when using 4 M glycerol and at a pH of 4.0, and graph (iii) shows the results when using 0.5 M glycerol and at a pH of 4.5. The absorbance of 50 mM PIPES buffer (pH 7.0) was measured as a control (indicated as “Buffer” in the figure).

Results

From FIG. 1(i), it was found that when the reaction was carried out using 4 M glycerol and at a pH of 5.0, the phosphorylation of glycerol using the phosphorylating enzymes including the respective amino acid sequences set forth in SEQ ID NOs: 3 to 9, 104, and 105 resulted in a significantly high phosphorylation efficiency, relative to the other phosphorylating enzymes. It was also found that among these phosphorylating enzymes, the phosphorylating enzyme including the amino acid sequence set forth in SEQ ID NO: 4 produced a particularly high amount of glycerol-3-phosphate and has an enhanced phosphorylation action on glycerol under these conditions. From these results, it turned out that a class A acid phosphatase having as the active center an amino acid sequence represented by the sequence (1) as described above can be utilized as a glycerol-phosphorylating enzyme.

From FIG. 1(ii), it was found that when the reaction was carried out using 4 M glycerol and at a pH of 4.0, the phosphorylation of glycerol using the phosphorylating enzyme including the amino acid sequence set forth in SEQ ID NO: 3, 4, 8, or 9, resulted in a significantly high phosphorylation efficiency, relative to the other phosphorylating enzymes. It was also found that among these phosphorylating enzymes, the phosphorylating enzyme including the amino acid sequence set forth in SEQ ID NO: 4 produced a particularly high amount of glycerol-3-phosphate and has an enhanced phosphorylation action on glycerol under these conditions.

From FIG. 1(iii), it was found that when the reaction was carried out using 0.5 M glycerol and at a pH of 4.5, the phosphorylation of glycerol using the phosphorylating enzyme including the amino acid sequence set forth in SEQ ID NO: 3, 5, or 9, resulted in high phosphorylation efficiency, relative to the other phosphorylating enzymes. It was found that in particular, the phosphorylation of glycerol using the phosphorylating enzyme including the amino acid sequence set forth in SEQ ID NO: 3 resulting in significantly high phosphorylation efficiency.

### Experimental Example 2

### Examination of Phosphate Group Donors

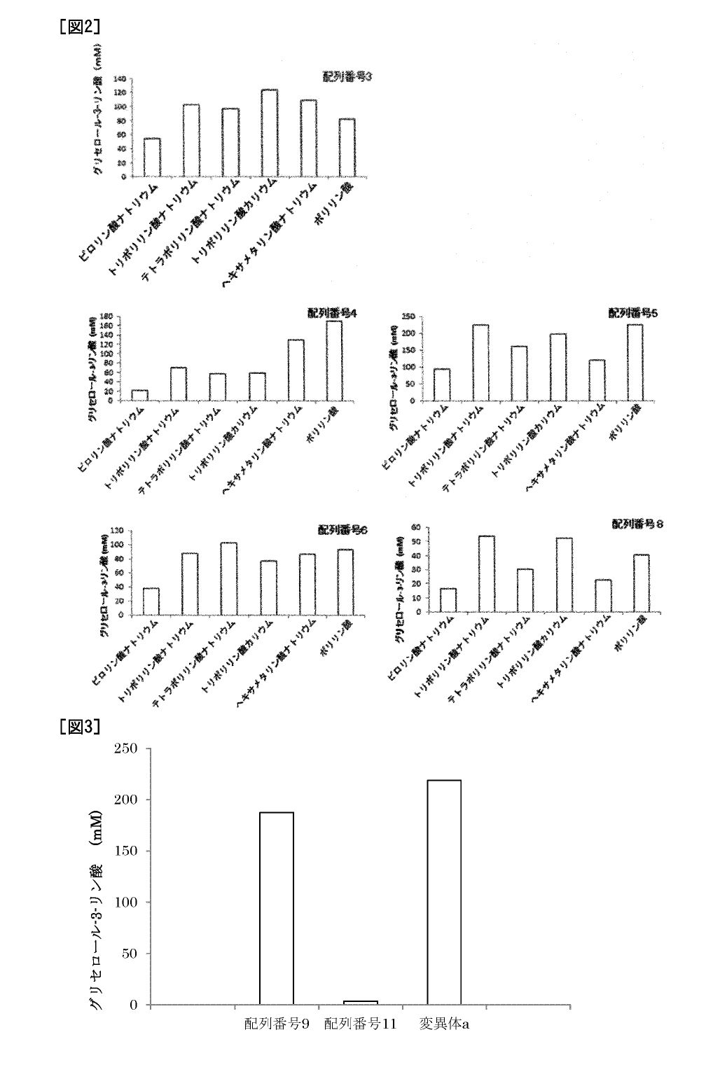

The efficiency of the phosphorylation of glycerol by phosphorylating enzymes (SEQ ID NOs: 3 to 6 and 8) was examined by changing the type of phosphate group donor. As a phosphate group donor, use was made of sodium pyrophosphate, sodium tripolyphosphate, sodium tetrapolyphosphate, potassium tripolyphosphate, sodium hexametaphosphate, or polyphosphoric acid. The polyphosphoric acid used was a polyphosphoric acid manufactured by Nacalai Tesque, Inc. The concentration of a phosphate group donor was set to be 0.2 M, except that the polyphosphoric acid was set to be at 7.5% by weight. A phosphorylating enzyme (phosphorylating enzyme set forth in SEQ ID NO: 3 to 6 or 8; 0.02 mg/mL each), a substrate (glycerol; at a concentration of 1 M), and a phosphate group donor were added to ultrapure water to prepare a reaction solution. The pH of the reaction solution was adjusted to a pH of 5.0 with potassium hydroxide or acetic acid. The reaction was carried out at 37° C. for 24 hours. After the reaction was completed, the amount of glycerol-3-phosphate produced was measured by an enzymatic method using glycerol-3-phosphate dehydrogenase as in Experimental Example 1. The results are shown in FIG. 2.

As shown in FIG. 2, it was found that the phosphorylated glycerol was obtained when any of the phosphate group donors was used. Particularly, the phosphorylating enzyme set forth in SEQ ID NO: 3 allowed the production of the phosphorylated product with higher efficiency by using sodium tripolyphosphate, sodium tetrapolyphosphate, potassium tripolyphosphate, sodium hexametaphosphate, or polyphosphoric acid as the phosphate group donor.

The phosphorylating enzyme set forth in SEQ ID NO: 4 allowed the production of the phosphorylated product with higher efficiency by using sodium hexametaphosphate or polyphosphoric acid as the phosphate group donor.

The phosphorylating enzyme set forth in SEQ ID NO: 5 allowed the production of the phosphorylated product with higher efficiency by using sodium tripolyphosphate, potassium tripolyphosphate, or polyphosphoric acid as the phosphate group donor.

The phosphorylating enzyme set forth in SEQ ID NO: 6 allowed the production of the phosphorylated product with higher efficiency by using sodium tripolyphosphate, sodium tetrapolyphosphate, potassium tripolyphosphate, sodium hexametaphosphate, or polyphosphoric acid as the phosphate group donor.

The phosphorylating enzyme set forth in SEQ ID NO: 8 allowed the production of the phosphorylated product with higher efficiency by using sodium tripolyphosphate, potassium tripolyphosphate, or polyphosphoric acid as the phosphate group donor.

### Experimental Example 3

### Phosphorylation of Glycerol by Phosphorylating Enzyme Mutants

(3-1) A Mutant Having Mutations Introduced in the Vicinity of the Active Center

(Introduction of Mutations and Generation of Transformants)

Mutations were introduced into a phosphorylating enzyme including the amino acid sequence set forth in SEQ ID NO: 11 that did not have any detectable activity to phosphorylate glycerol, to generate an enzyme mutant, Mutant a. The generation procedure is indicated below.

To obtain Mutant a, PCR reactions were performed using as a template a base sequence encoding the amino acid sequence set forth in SEQ ID NO: 11, and combinations of the primers indicated in Table 3. The resulting PCR product was digested with the restriction enzymes described corresponding to the column of Mutant in Table 3. Ligation was performed between a pET22b(+) vector (Novagen) treated with the same restriction enzymes as those with which the PCR product had been treated and the restriction enzyme-treated PCR product, to obtain a vector for expression of the encoded glycerol-phosphorylating enzyme. The vector further carry, besides the base sequence encoding Mutant a, a T7 promoter, a pelB signal for transport of the expressed protein into the periplasm, a His-tag gene for protein purification, and an ampicillin resistance gene for selection with a drug. The vector obtained by the above-described procedures was transformed into cells of an Escherichia coli (E. coli) BL21(DE3) strain (Novagen) by heat shock method.

The primer sets and restriction enzyme sites used for the generation of Mutant a, and the base sequences of the primers are indicated in Tables 3 and 4, respectively. In Table 3, “F144G/A151T” in Mutant a, in the column of Mutant, indicates that the phenylalanine residue at position 144 in SEQ ID NO: 11 was replaced with a glycine residue and the alanine residue at position 151 was replaced with a threonine residue.

TABLE 3

| SEQ ID NO: of (the | | | |
|---|---|---|---|
| whole amino acid | | Primer | Restriction |
| sequence of) mutant | Mutant | set | enzyme site |
| 13 | a | Primers | NcoI and BamHI; |
| | (F144G/ | (1) and (4); | BamHI and HindIII |
| | A151T) | Primers | |
| | | (3) and (2) | |

TABLE 4

| Primer | | |
|---|---|---|
| SEQ ID | Primer | |
| NO: | No. | Sequence (5′ to 3′) |
| 62 | (1) | TACGTTCCATGGATTCCGC |
| | | CCCCTCGCTCGAGAAGGA |
| 63 | (2) | ATCCAAGCTTGAGCAGGGG |
| | | CTGGCGCGCGGCCGCAT |
| 64 | (3) | AGCGGATCCTATCCCTCCG |
| | | GTCATACTAGCCAGGGG |
| 65 | (4) | ATAGGATCCGCTGTCCGCG |
| | | AGTTCGGCCGTGCG |

*In Table 4, the underlined sequences indicate a restriction enzyme cleavage site.

(Culturing of Transformants)

Culturing of transformant cells of an E. coli BL21(DE3) strain carrying the vector for expression of the encoded glycerol-phosphorylating enzyme was performed by incubating them at a culture temperature of 27° C. in an LB medium (manufactured by Nacalai Tesque, Inc. and having a composition of 1% by weight of tryptone, 0.5% by weight of dry yeast extract, 0.5% by weight of sodium chloride) containing 0.2% by weight of glucose, 0.5% by weight of casamino acid, and 0.1 mg/mL of ampicillin. When the optical density of the culture at 600 nm reached 0.5, isopropyl-β-thiogalactopyranoside was added to be 0.1 mM and additional culturing was performed at a culture temperature of 27° C. for a period of 16 to 24 hours.

(Obtaining of a Glycerol-Phosphorylating Enzyme Mutant)

After the culture was completed, 15 g of wet cells was suspended in 20 mM Tris-HCl buffer (pH 8.0) containing 150 mM sodium chloride, and then subjected to an ultrasonic homogenizer (TOMY UD201) to obtain a cell extract solution. The cell extract solution was subjected to centrifugation at 20,000×g for 20 minutes to collect the supernatant fraction. To the supernatant, ammonium sulfate was added to a saturation of 60%, followed by centrifugation at 20,000×g for 20 minutes to collect a precipitate. The precipitate after the ammonium sulfate fractionation was suspended in 20 mM Tris-HCl buffer (pH 7.5), and then dialyzed against 20 mM Tris-HCl buffer (pH 7.5) containing 50 mM sodium chloride. After the dialysis was completed, affinity purification was carried out using a His-Trap HP column. Fractions containing the glycerol-phosphorylating enzyme were collected, and then subjected to dialysis against 20 mM Tris-HCl buffer (pH 7.5) to obtain the glycerol-phosphorylating enzyme mutant. The amino acid sequences of the active center of the phosphorylating enzyme including the amino acid sequence set forth in SEQ ID NO: 11 and the enzyme mutant, Mutant a, are indicated in Table 5.

TABLE 5

| | Amino acid sequence |
|---|---|
| | of the active center |
| SEQ ID NO: 11 | Phe Ser Tyr Pro Ser Gly His Ala |
| Mutant a | Gly Ser Tyr Pro Ser Gly His Thr |
| (F144G/A151T) | |

Purification Conditions

Conditions for the Affinity Purification were as Follows.

Column: His-Trap HP (manufactured by GE healthcare)
Equilibration buffer: 20 mM Tris-HCl (pH 7.5), 50 mM sodium chloride
Elution buffer: 20 mM Tris-HCl (pH 7.5), 50 mM sodium chloride, 1 M imidazole (elution in a gradient of the elution buffer from 0 to 50%)
Flow rate: 5.0 mL/min
Purification equipment: AKTA prime plus (GE healthcare)

(Phosphorylation of Glycerol Using a Glycerol-Phosphorylating Enzyme Mutant, Mutant a)

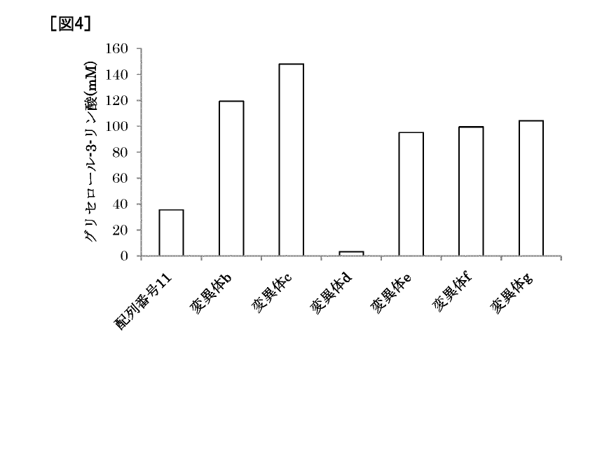

The glycerol phosphorylating reaction was performed using Mutant a, which is the glycerol-phosphorylating enzyme obtained as described above. To ultrapure water were added glycerol (4 M) and a polyphosphoric acid as a phosphate group donor (7.5% by weight; manufactured by Nacalai Tesque, Inc. under a trade name of polyphosphoric acid), to prepare a reaction mixture solution. The pH of the reaction solution was adjusted to a pH of 5.0 with KOH. The phosphorylating enzyme, Mutant a, was added to the reaction mixture solution to make a final concentration of 50 μg/mL. The reaction solution was subjected to reaction in a temperature-controlled air incubator at 37° C. for 24 hours. The concentration of glycerol-3-phosphate in the sample was detected by an enzymatic method using glycerol-3-phosphate dehydrogenase. The phosphorylation of glycerol and detection of glycerol-3-phosphate were carried out as in Experimental Example 1. The results are shown in FIG. 3.

As shown in FIG. 3, it was observed that Mutant a having F144G/A151T mutations introduced in the phosphorylating enzyme set forth in SEQ ID NO: 11 that had an extremely low level of phosphorylating enzyme activity resulted in a significant improvement in the enzyme activity.

(3-2) Mutants Having Mutations Introduced in the Vicinity of the Active Center—

Mutations were introduced into a phosphorylating enzyme including the amino acid sequence set forth in SEQ ID NO: 11 that did not have any detectable activity to phosphorylate glycerol, to generate enzyme mutants, Mutants b to g. Generation procedures for Mutants b to g were as described above in (3-1), except for using as a template a base sequence (set forth in SEQ ID NO: 128) in which a (wild-type) base sequence encoding the amino acid sequence set forth in SEQ ID NO: 11 was codon optimized for expression in E. coli and using the primers indicated in Tables 6 and 7.

TABLE 6

| SEQ ID NO: of (the | | | |
|---|---|---|---|
| whole amino acid | | Primer | Restriction |
| sequence of) mutant | Mutant | set | enzyme site |
| 116 | b | Primers | NcoI and NheI; |
| | (F144A) | (5) and (7); | NheI and HindIII |
| | | Primers | |
| | | (8) and (6) | |
| 117 | c | Primers | NcoI and BamHI; |
| | (F144G) | (5) and (9); | BamHI and HindIII |
| | | Primers | |
| | | (10) and (6) | |
| 118 | d | Primers | NcoI and NdeI; |
| | (A151D) | (5) and (11); | NdeI and HindIII |
| | | Primers | |
| | | (12) and (6) | |
| 119 | e | Primers | NcoI and NdeI; |
| | (A151T) | (5) and (13); | NdeI and HindIII |
| | | Primers | |
| | | (11) and (6) | |
| 120 | f | Primers | NcoI and NdeI; |
| | (A151S) | (5) and (14); | NdeI and HindIII |
| | | Primers | |
| | | (15) and (6) | |
| 121 | g | Primers | NcoI and BamHI; |
| | (F144G/ | (5) and (9); | BamHI and HindIII |
| | A151S) | Primers | |
| | | (15) and (6) | |

TABLE 7

| Primer | | |
|---|---|---|
| SEQ ID | Primer | |
| NO: | No. | Sequence (5′ to 3′) |
| 129 | (5) | GGCGATGGCCATGGACAGTGCCCC |
| | | GAGTCTGGAA |
| 130 | (6) | TAAAGAAGCTTCAGCAGCGGTTGA |
| | | CGGGCAGC |
| 131 | (7) | ACTCGGATAGCTAGCGCTGTCCGC |
| | | CAGTTCAGC |
| 132 | (8) | GCGGACAGCGCTAGCTATCCGAGT |
| | | GGTCATGCGTCCCAG |
| 133 | (9) | AATAGGATCCGCTGTCCGCCAGTT |
| | | CAGCGGT |
| 134 | (10) | GACAGAGGATCCTATCCGAGTGGT |
| | | CATGCGTCCCAGGGT |
| 135 | (11) | GATCAGACCATATGCCCAACCCTG |
| | | GGAGTCATGACCACT |
| 136 | (12) | CAGGGTTGGGCATATGGTCTGATC |
| | | ATGGCAAAC |
| 137 | (13) | GATCAGACCATATGCCCAACCCTG |
| | | GGACGTATGACCACT |
| 138 | (14) | GATCAGACCATATGCCCAACCCTG |
| | | GGAGCTATGACCACT |
| 139 | (15) | GACAGAGGATCCTATCCGAGTGGT |
| | | CATTCGTCCCAGGGT |

The amino acid sequences of the active center of the phosphorylating enzyme including the amino acid sequence set forth in SEQ ID NO: 11 and the enzyme mutants, Mutants b to g, are indicated in Table 8.

TABLE 8

| | Amino acid sequence |
|---|---|
| | of the active center |
| SEQ ID NO: 11 | Phe Ser Tyr Pro Ser Gly His Ala |
| Mutant b | Ala Ser Tyr Pro Ser Gly His Ala |
| (F144A) | |
| Mutant c | Gly Ser Tyr Pro Ser Gly His Ala |
| (F144G) | |
| Mutant d | Phe Ser Tyr Pro Ser Gly His Asp |
| (A151D) | |
| Mutant e | Phe Ser Tyr Pro Ser Gly His Thr |
| (A151T) | |
| Mutant f | Phe Ser Tyr Pro Ser Gly His Ser |
| (A151S) | |
| Mutant g | Gly Ser Tyr Pro Ser Gly His Ser |
| (F144G/A151S) | |

Culturing of transformants, obtaining of glycerol-phosphorylating enzyme mutants, and phosphorylation of glycerol were performed in the same conditions as those described above in (3-1).

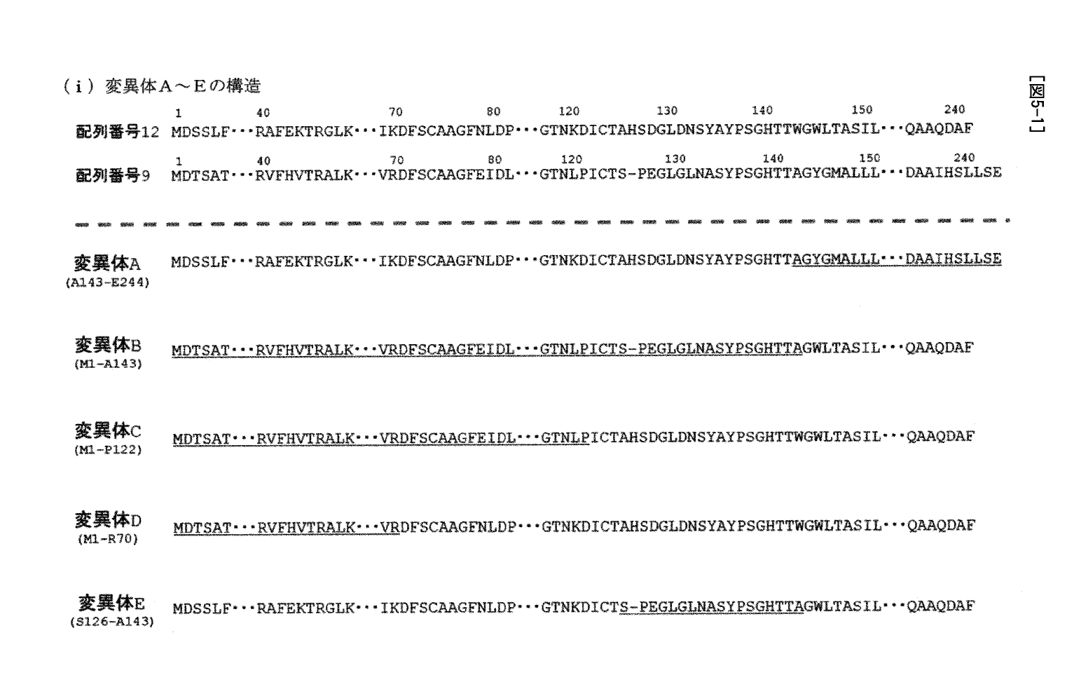

The results obtained are shown in FIG. 4. From these results, it was observed that Mutant d had little detectable activity to phosphorylate glycerol, while Mutants b, c, and e to g exhibited a superior activity to phosphorylate glycerol. Also from these results, it turned out that a class A acid phosphatase having as the active center an amino acid sequence represented by the sequence (1) as described above can be utilized as a glycerol-phosphorylating enzyme.

(3-2) Mutants Having Mutations Introduced in an N-Terminal Portion

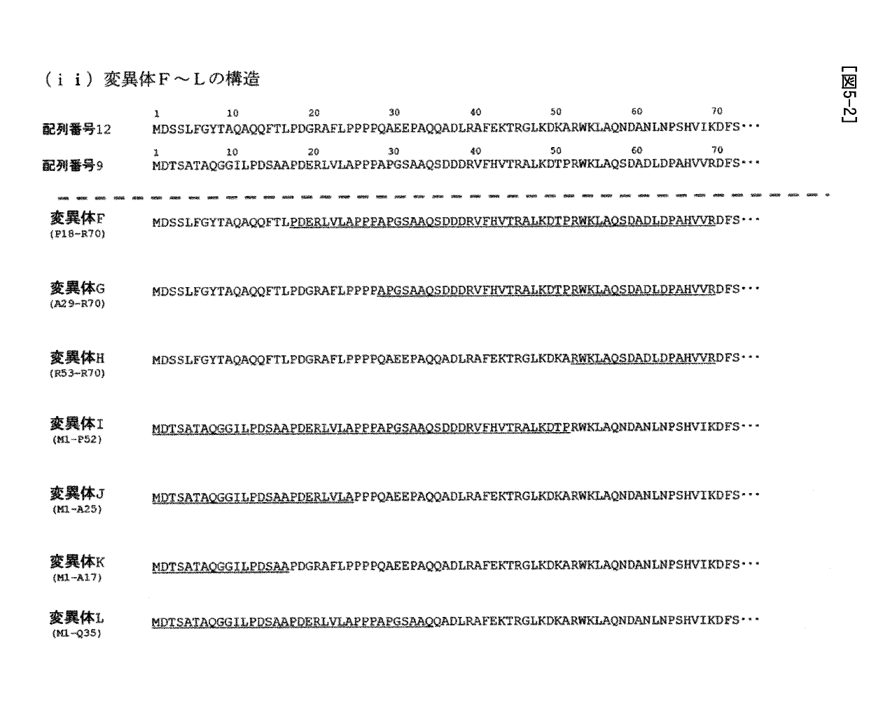

Mutants A to L were generated in which an N-terminal portion of the amino acid sequence set forth in SEQ ID NO: 12 was replaced with a partial sequence comprised of the amino acid sequence set forth in SEQ ID NO: 9, by means of using as a template a DNA sequence encoding the amino acid sequence set forth in SEQ ID NO: 12. Generation procedures were as in Mutants a to d described above in (3-1), and PCR products were obtained using the primer sets and template indicated below in Table 9. The PCR product was treated with restriction enzymes corresponding to the restriction enzyme sites described for the respective mutants in Table 9, and inserted into a vector. The structures of Mutants A to L are shown in FIGS. 5(i) and (ii).

Combinations of the primer sets, restriction enzyme sites, and templates used for the generation of Mutants A to L having mutations introduced in an N-terminal portion are shown in Table 9, and the base sequences of the primers are shown in Table 10. In Table 9, the description “A143-E244” in Mutant A, for example, in the column of Mutant, indicates that the amino acid sequence corresponding to positions 144 to 242 in SEQ ID NO: 12 was replaced with the corresponding amino acid sequence of from the alanine at position 143 to the glutamic acid at position 244 in SEQ ID NO: 9.

TABLE 9

| SEQ ID | | | | |
|---|---|---|---|---|
| NO: of | | Primer | Restriction | Template |
| mutant | Mutant | set | enzyme site | used |
| 14 | A | Primers | NcoI and PstI; | a plasmid encoding |
| | (A143- | (1) and (6); | PstI and HindIII | SEQ ID NO: 12; |
| | E244) | Primers | | a plasmid encoding |
| | | (5) and (4) | | SEQ ID NO: 9 |
| 15 | B | Primers | NcoI and PstI; | a plasmid encoding |
| | (M1- | (3) and (8); | PstI and HindIII | SEQ ID NO: 9; |
| | A143) | Primers | | a plasmid encoding |
| | | (7) and (2) | | SEQ ID NO: 12 |
| 16 | C | Primers | NcoI and NheI; | a plasmid encoding |
| | (M1- | (3) and (10); | NheI and HindIII | SEQ ID NO: 9; |
| | P122) | Primers | | a plasmid encoding |
| | | (9) and (2) | | SEQ ID NO: 12 |
| 17 | D | Primers | NcoI and SalI; | a plasmid encoding |
| | (M1- | (3) and (12); | XhoI and HindIII | SEQ ID NO: 9; |
| | R70) | Primers | | a plasmid encoding |
| | | (11) and (2) | | SEQ ID NO: 12 |
| 18 | E | Primers | NcoI and SpeI; | a plasmid encoding |
| | (S126- | (1) and (14); | SpeI and HindIII | SEQ ID NO: 9; |
| | A143) | Primers | | a plasmid encoding |
| | | (13) and (2) | | SEQ ID NO: 12 |
| 19 | F | Primers | NcoI and SpeI; | a plasmid encoding |
| | (P18- | (1) and (16); | SpeI and HindIII | SEQ ID NO: 9; |
| | R70) | Primers | | a plasmid encoding |
| | | (15) and (2) | | Mutant D |
| 20 | G | Primers | NcoI and PstI; | a plasmid encoding |
| | (A29- | (1) and (18); | PstI and HindIII | SEQ ID NO: 9; |
| | R70) | Primers | | a plasmid encoding |
| | | (17) and (2) | | Mutant D |
| 21 | H | Primers | NcoI and NheI; | a plasmid encoding |
| | (R53- | (1) and (20); | NheI and HindIII | SEQ ID NO: 9; |
| | R70) | Primers | | a plasmid encoding |
| | | (19) and (2) | | Mutant D |
| 22 | I | Primers | NcoI and NheI; | a plasmid encoding |
| | (M1- | (3) and (22); | NheI and HindIII | SEQ ID NO: 9; |
| | P52) | Primers | | a plasmid encoding |
| | | (21) and (2) | | SEQ ID NO: 12 |
| 23 | J | Primers | NcoI and NheI; | a plasmid encoding |
| | (M1- | (3) and (24); | NheI and HindIII | SEQ ID NO: 9; |
| | A25) | Primers | | a plasmid encoding |
| | | (23) and (2) | | SEQ ID NO: 12 |
| 24 | K | Primers | NcoI and PstI; | a plasmid encoding |
| | (M1- | (3) and (26); | PstI and HindIII | SEQ ID NO: 9; |
| | A17) | Primers | | a plasmid encoding |
| | | (25) and (2) | | SEQ ID NO: 12 |
| 25 | L | Primers | NcoI and PstI; | a plasmid encoding |
| | (M1- | (3) and (28); | PstI and HindIII | SEQ ID NO: 9; |
| | Q35) | Primers | | a plasmid encoding |
| | | (27) and (2) | | SEQ ID NO: 12 |

TABLE 10

| Primer | | |
|---|---|---|
| SEQ | Primer | |
| ID NO: | No. | Sequence (5′ to 3′) |
| 66 | 1 | CTTAGACCATGGATAGCAGCCTTTTTGGATATACC |
| 67 | 2 | AAGTAAGCTTAAAAGCGTCTTGCGCAGCCTGCTGTTC |
| 68 | 3 | TAACGCCCATGGATACGTCCGCTACCGCCCAAGGCGGC |
| 67 | 4 | AACAAAGCTTTTCGCTGAGCAGGGAATGGATCGCGGC |
| 70 | 5 | GACTGCAGGCTACGGGATGGCGCTGCTCCTG |
| 71 | 6 | ATCCTGCAGTTGTATGGCCAGAGGGATA |
| 72 | 7 | CAACTGCAGGATGGCTCACGGCATCCATT |
| 73 | 8 | TCCTGCAGTCGTATGACCCGACGGATAGGACGC |
| 74 | 9 | AATAAACACATATGCACAGCACATTCAGATGGGCTT |
| 75 | 10 | GAGAGCTGCATATGGGCAGGTTCGTACCCACGAACG |
| 76 | 11 | TTTAATGTCGACCCAGAAAAACTTCCCGCAATG |
| 77 | 12 | GCCAGCTCGAGATTGAAACCCGCCGCACAGGAGAA |
| 78 | 13 | ACACTAGTCCGGAAGGTCTTGGTCTCAACGCTTCCTAT |
| | | CCCTCT |
| 79 | 14 | GGACTAGTGCAGATGTCTTTATTTGTGCCGAC |
| 80 | 15 | CGACTAGTTCTGGCTCCGCCGCCGGCG |
| 81 | 16 | AAACTAGTCGTTCATCAGGTAGGGTAAACTG |
| 82 | 17 | GGAACTGCAGCGCAGAGTGATGACGACCGGGTG |
| 83 | 18 | AGTGCTGCAGATCCGGGCGCTGGTGGCGGGGGCAAAA |
| 84 | 19 | CGCTGGAAGCTAGCGCAGAGCGATGCGGATCTG |
| 85 | 20 | ATTCTGTAGCTAGCTTCCAGCGCGCCTTATCTTTTAA |
| 86 | 21 | CGCTGGAAGCTAGCCCAGAATGACGCCAACCTT |
| 87 | 22 | GCTCTGAGCTAGCTTCCAGCGCGGCGTGTCCTTCAG |
| 88 | 23 | GACTTCAGCTAGCACCGCCACCACAGGCAGAAGAACCC |
| 89 | 24 | CGGCGGAGCTAGCACGAGACGCTCGTCCGGAGCCGC |
| 90 | 25 | CAGTCTGCAGCACCTGATGGACGTGCATTTTTG |
| 91 | 26 | GGAGCTGCAGAGTCCGGCAGGATGCCGCCTTG |
| 92 | 27 | AACCTGCAGCACAACAGGCAGACTTGCGGGCT |
| 93 | 28 | CTGCTGCAGATCCGGGCGCCGGCGGCGGAGC |

(Phosphorylation of Glycerol Using Glycerol-Phosphorylating Enzyme Mutants, Mutants A to L)

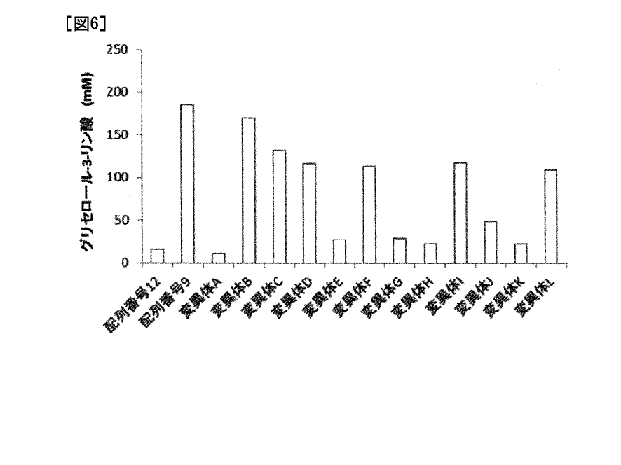

The glycerol phosphorylating reaction was performed using the resulting Mutants A to L. The phosphorylation of glycerol was carried out as described above for Mutants a to g, and the detection of glycerol-3-phosphate was carried out as in Experimental Example 1. The results are shown in FIG. 6.

As shown in FIG. 5, the glycerol phosphorylating reactions in which Mutants A to L were used resulted in an increased amount of produced glycerol-3-phosphate for Mutants B, C, and D. Based on these results, it was revealed that the amino acid sequence of the amino acids at positions 1 to 70 from the N terminus of the amino acid sequence set forth in SEQ ID NO: 9 is responsible for the activity of the glycerol-phosphorylating enzyme. In addition, it was found that the glycerol phosphorylating reactions in which Mutants F to L were used resulted in an improvement in the activity of the phosphorylating enzyme for Mutants F, I, and L. The other Mutants G, H, J, and K, on the other hand, did not result in a great improvement in the activity of the phosphorylating enzyme, as compared to Mutants F and I. Based on these results, it was revealed that the amino acid sequence of from the proline residue at position 18 to the glutamine residue at position 35 from the N terminus of the amino acid sequence set forth in SEQ ID NO: 9 is responsible for the activity of the glycerol-phosphorylating enzyme.

Therefore, it was revealed that the amino acid sequence set forth in SEQ ID NO: 2, PDERLVLAPPPAPGSAAQ, is important for the expression of the enzyme activity of the above-described enzymes catalyzing the glycerol phosphorylating reaction.

### Sequence Listing Free Text

SEQ ID NO: 1 represents the amino acid sequence represented by sequence

SEQ ID NO: 2 represents the amino acid sequence of the N-terminal essential region of the phosphorylating enzyme.

SEQ ID NO: 13 represents the amino acid sequence of Mutant a (F144G/A151T).

SEQ ID NO: 14 represents the amino acid sequence of Mutant A (A143-E244).

SEQ ID NO: 15 represents the amino acid sequence of Mutant B (M1-A143).

SEQ ID NO: 16 represents the amino acid sequence of Mutant C (M1-P122).

SEQ ID NO: 17 represents the amino acid sequence of Mutant D (M1-R70).

SEQ ID NO: 18 represents the amino acid sequence of Mutant E (S126-A143).

SEQ ID NO: 19 represents the amino acid sequence of Mutant F (P18-R70).

SEQ ID NO: 20 represents the amino acid sequence of Mutant G (A29-R70).

SEQ ID NO: 21 represents the amino acid sequence of Mutant H (R53-R70).

SEQ ID NO: 22 represents the amino acid sequence of Mutant I (M1-P52).

SEQ ID NO: 23 represents the amino acid sequence of Mutant J (M1-A25).

SEQ ID NO: 24 represents the amino acid sequence of Mutant K (M1-A17).

SEQ ID NO: 25 represents the amino acid sequence of Mutant L (M1-Q35).

SEQ ID NO: 42 represents a forward primer for the phosphorylating enzyme set forth in amino acid sequence 3.

SEQ ID NO: 43 represents a reverse primer for the phosphorylating enzyme set forth in amino acid sequence 3.

SEQ ID NO: 44 represents a forward primer for the phosphorylating enzyme set forth in amino acid sequence 4.

SEQ ID NO: 45 represents a reverse primer for the phosphorylating enzyme set forth in amino acid sequence 4.

SEQ ID NO: 46 represents a forward primer for the phosphorylating enzyme set forth in amino acid sequence 5.

SEQ ID NO: 47 represents a reverse primer for the phosphorylating enzyme set forth in amino acid sequence 5.

SEQ ID NO: 48 represents a forward primer for the phosphorylating enzyme set forth in amino acid sequence 6.

SEQ ID NO: 49 represents a reverse primer for the phosphorylating enzyme set forth in amino acid sequence 6.

SEQ ID NO: 50 represents a forward primer for the phosphorylating enzyme set forth in amino acid sequence 7.

SEQ ID NO: 51 represents a reverse primer for the phosphorylating enzyme set forth in amino acid sequence 7.

SEQ ID NO: 52 represents a forward primer for the phosphorylating enzyme set forth in amino acid sequence 8.

SEQ ID NO: 53 represents a reverse primer for the phosphorylating enzyme set forth in amino acid sequence 8.

SEQ ID NO: 54 represents a forward primer for the phosphorylating enzyme set forth in amino acid sequence 9.

SEQ ID NO: 55 represents a reverse primer for the phosphorylating enzyme set forth in amino acid sequence 9.

SEQ ID NO: 56 represents a forward primer for the phosphorylating enzyme set forth in amino acid sequence 10.

SEQ ID NO: 57 represents a reverse primer for the phosphorylating enzyme set forth in amino acid sequence 10.

SEQ ID NO: 58 represents a forward primer for the phosphorylating enzyme set forth in amino acid sequence 11.

SEQ ID NO: 59 represents a reverse primer for the phosphorylating enzyme set forth in amino acid sequence 11.

SEQ ID NO: 60 represents a forward primer for the phosphorylating enzyme set forth in amino acid sequence 12.

SEQ ID NO: 61 represents a reverse primer for the phosphorylating enzyme set forth in amino acid sequence 12.

SEQ ID NO: 62 represents the base sequence of primer number (1).

SEQ ID NO: 63 represents the base sequence of primer number (2).

SEQ ID NO: 64 represents the base sequence of primer number (3).

SEQ ID NO: 65 represents the base sequence of primer number (4).

SEQ ID NO: 66 represents the base sequence of primer number 1.

SEQ ID NO: 67 represents the base sequence of primer number 2.

SEQ ID NO: 68 represents the base sequence of primer number 3.

SEQ ID NO: 69 represents the base sequence of primer number 4.

SEQ ID NO: 70 represents the base sequence of primer number 5.

SEQ ID NO: 71 represents the base sequence of primer number 6.

SEQ ID NO: 72 represents the base sequence of primer number 7.

SEQ ID NO: 73 represents the base sequence of primer number 8.

SEQ ID NO: 74 represents the base sequence of primer number 9.

SEQ ID NO: 75 represents the base sequence of primer number 10.

SEQ ID NO: 76 represents the base sequence of primer number 11.

SEQ ID NO: 77 represents the base sequence of primer number 12.

SEQ ID NO: 78 represents the base sequence of primer number 13.

SEQ ID NO: 79 represents the base sequence of primer number 14.

SEQ ID NO: 80 represents the base sequence of primer number 15.

SEQ ID NO: 81 represents the base sequence of primer number 16.

SEQ ID NO: 82 represents the base sequence of primer number 17.

SEQ ID NO: 83 represents the base sequence of primer number 18.

SEQ ID NO: 84 represents the base sequence of primer number 19.

SEQ ID NO: 85 represents the base sequence of primer number 20.

SEQ ID NO: 86 represents the base sequence of primer number 21.

SEQ ID NO: 87 represents the base sequence of primer number 22.

SEQ ID NO: 88 represents the base sequence of primer number 23.

SEQ ID NO: 89 represents the base sequence of primer number 24.

SEQ ID NO: 90 represents the base sequence of primer number 25.

SEQ ID NO: 91 represents the base sequence of primer number 26.

SEQ ID NO: 92 represents the base sequence of primer number 27.

SEQ ID NO: 93 represents the base sequence of primer number 28.

SEQ ID NO: 116 represents the amino acid sequence of Mutant b (F144A).

SEQ ID NO: 117 represents the amino acid sequence of Mutant c (F144G).

SEQ ID NO: 118 represents the amino acid sequence of Mutant d (A151D).

SEQ ID NO: 119 represents the amino acid sequence of Mutant e (A151T).

SEQ ID NO: 120 represents the amino acid sequence of Mutant f (A151S).

SEQ ID NO: 121 represents the amino acid sequence of Mutant g (F144G/A151S).

SEQ ID NO: 124 represents a forward primer for the phosphorylating enzyme set forth in amino acid sequence 104.

SEQ ID NO: 125 represents a reverse primer for the phosphorylating enzyme set forth in amino acid sequence 104.

SEQ ID NO: 126 represents a forward primer for the phosphorylating enzyme set forth in amino acid sequence 105.

SEQ ID NO: 127 represents a reverse primer for the phosphorylating enzyme set forth in amino acid sequence 105.

SEQ ID NO: 128 represents a base sequence in which a gene encoding the phosphorylating enzyme set forth in SEQ ID NO: 11 was codon optimized for expression in Escherichia coli.

SEQ ID NO: 129 represents the base sequence of primer number (5).

SEQ ID NO: 130 represents the base sequence of primer number (6).

SEQ ID NO: 131 represents the base sequence of primer number (7).

SEQ ID NO: 132 represents the base sequence of primer number (8).

SEQ ID NO: 133 represents the base sequence of primer number (9).

SEQ ID NO: 134 represents the base sequence of primer number (10).

SEQ ID NO: 135 represents the base sequence of primer number (11).

SEQ ID NO: 136 represents the base sequence of primer number (12).

SEQ ID NO: 137 represents the base sequence of primer number (13).

SEQ ID NO: 138 represents the base sequence of primer number (14).

SEQ ID NO: 139 represents the base sequence of primer number (15).

1. A method for production of a phosphorylated glycerol, comprising a step of allowing a phosphorylating enzyme to act on glycerol in the presence of a phosphate group donor,
wherein the phosphorylating enzyme is a class A acid phosphatase and comprises a polypeptide comprising as an active center an amino acid sequence represented by the sequence (1):
-X¹-X²-X³-Pro-Ser-Gly-His-X⁴- (1)
wherein,
X¹ denotes glycine, alanine, or phenylalanine;
X² denotes any amino acid;
X³ denotes tyrosine or tryptophan; and
X⁴ denotes threonine, serine, or alanine;
with the proviso that when X¹ is phenylalanine, X⁴ is threonine or serine.

2. The method for production according to claim 1, wherein X¹ and X⁴ in the sequence (1) are any of (A1) to (A8) below:
(A1) X¹ being glycine and X⁴ being threonine;
(A2) X¹ being glycine and X⁴ being serine;
(A3) X¹ being alanine and X⁴ being serine;
(A4) X¹ being alanine and X⁴ being threonine;
(A5) X¹ being alanine and X⁴ being alanine;
(A6) X¹ being glycine and X⁴ being alanine;
(A7) X¹ being phenylalanine and X⁴ being threonine; and
(A8) X¹ being phenylalanine and X⁴ being serine.

3. The method for production according to claim 1, wherein in the sequence (1), X² is serine, alanine, or aspartic acid.

4. The method for production according to claim 1, wherein X¹, X², X³, and X⁴ in the sequence (1) are any of (B1) to (B11) below:
(B1) X¹ being glycine, X² being serine, X³ being tyrosine, and X⁴ being threonine;
(B2) X¹ being glycine, X² being alanine, X³ being tyrosine, and X⁴ being threonine;
(B3) X¹ being glycine, X² being serine, X³ being tyrosine, and X⁴ being serine;
(B4) X¹ being glycine, X² being aspartic acid, X³ being tyrosine, and X⁴ being threonine;
(B5) X¹ being alanine, X² being serine, X³ being tyrosine, and X⁴ being serine;
(B6) X¹ being alanine, X² being serine, X³ being tyrosine, and X⁴ being threonine;
(B7) X¹ being alanine, X² being serine, X³ being tyrosine, and X⁴ being alanine;
(B8) X¹ being glycine, X² being serine, X³ being tyrosine, and X⁴ being alanine;
(B9) X¹ being phenylalanine, X² being serine, X³ being tyrosine, and X⁴ being threonine;
(B10) X¹ being phenylalanine, X² being serine, X³ being tyrosine, and X⁴ being serine; and
(B11) X¹ being glycine, X² being serine, X³ being tryptophan, and X⁴ being serine.

5. The method for production according to claim 1, wherein the histidine (His) residue at the 7-th position in the amino acid sequence represented by the sequence (1) is located between positions 120 and 160 from the N-terminus of the polypeptide employed as the phosphorylating enzyme.

6. The method for production according to claim 1, wherein the phosphorylating enzyme further comprises a polypeptide as defined in (I) or (II) below:
including a polypeptide as defined in (II);
(I) a polypeptide that has the amino acid sequence set forth in SEQ ID NO: 2, on the N-terminal side of a stretch of the amino acid sequence coding for the active center represented by the sequence (1), or
(II) a polypeptide that comprises, on the N-terminal side of a stretch of the amino acid sequence coding for the active center represented by the sequence (1), an amino acid sequence having one or a few amino acids substituted, deleted, inserted, or added in the amino acid sequence set forth in SEQ ID NO: 2 and has an activity to catalyze the glycerol phosphorylating reaction which is comparable or superior to that of the polypeptide as defined in (I).

7. The method for production according to claim 6, wherein the amino acid residue at the first position in the amino acid sequence set forth in SEQ ID NO: 2 is located between positions 5 and 40 from the N-terminus of the polypeptide employed as the phosphorylating enzyme.

8. The method for production according to claim 1, wherein the phosphorylating enzyme further comprises any of the polypeptides as defined in (III) to (VI) below:
(III) a polypeptide that has the amino acid sequence set forth in any of SEQ ID NOs: 26 to 32 and 106 to 110, on the N-terminal side of the amino acid sequence represented by the sequence (1);
(IV) a polypeptide that has, on the N-terminal side of the amino acid sequence represented by the sequence (1), an amino acid sequence having one or several amino acids substituted, deleted, inserted, or added in the amino acid sequence set forth in any of SEQ ID NOs: 26 to 32 and 106 to 110 and has an activity to catalyze the glycerol phosphorylating reaction which is comparable or superior to that of its corresponding polypeptide as defined in (III);
(V) a polypeptide that has the amino acid sequence set forth in any of SEQ ID NOs: 33 to 39 and 111 to 115, on the C-terminal side of the amino acid sequence represented by the sequence (1); and
(VI) a polypeptide that has, on the C-terminal side of the amino acid sequence represented by the sequence (1), an amino acid sequence having one or several amino acids substituted, deleted, inserted, or added in the amino acid sequence set forth in any of SEQ ID NOs: 33 to 39 and 111 to 115 and has an activity to catalyze the glycerol phosphorylating reaction which is comparable or superior to that of its corresponding polypeptide as defined in (V).

9. The method for production according to claim 1, wherein the phosphorylating enzyme comprises a polypeptide as defined in (VII) or (VIII) below:
(VII) a polypeptide that has the amino acid sequence set forth in any of SEQ ID NOs: 3 to 9, 13, 104, 105, 116, 117, and 119 to 121, or
(VIII) a polypeptide that comprises an amino acid sequence having, in the amino acid sequence set forth in any of SEQ ID NOs: 3 to 9, 13, 104, 105, 116, 117, and 119 to 121, one or several amino acids substituted, deleted, inserted, or added in a region outside the active center comprising the amino acid sequence represented by the sequence (1) and has an activity to catalyze the glycerol phosphorylating reaction which is comparable or superior to that of its corresponding polypeptide as defined in (VII).

10. A method for production of a phosphorylated glycerol, comprising a step of allowing a phosphorylating enzyme to act on glycerol in the presence of a phosphate group donor,
wherein the phosphorylating enzyme is a class A acid phosphatase and comprises a polypeptide as defined in (i) or (ii) below:
(i) a polypeptide that has the amino acid sequence set forth in SEQ ID NO: 2 and has an activity to catalyze the glycerol phosphorylating reaction, or
(ii) a polypeptide that has an amino acid sequence having one or several amino acids substituted, deleted, inserted, or added in the amino acid sequence set forth in SEQ ID NO: 2 and has an activity to catalyze the glycerol phosphorylating reaction which is comparable or superior to that of the polypeptide as defined in (i).

11. The method for production according to claim 10, wherein the amino acid residue at the first position in the amino acid sequence set forth in SEQ ID NO: 2 is located between positions 5 and 40 from the N-terminus of the polypeptide employed as the phosphorylating enzyme.

12. The method for production according to claim 10, wherein the phosphorylating enzyme comprises a polypeptide having, on the C-terminal side of the amino acid sequence set forth in SEQ ID NO: 2, an amino acid sequence represented by the sequence (1):
-X¹-X²-X³-Pro-Ser-Gly-His-X⁴- (1)
wherein,
X¹ denotes glycine, alanine, or phenylalanine;
X² denotes any amino acid;
X³ denotes tyrosine or tryptophan; and
X⁴ denotes threonine, serine, or alanine;
with the proviso that when X¹ is phenylalanine, X⁴ is threonine or serine.

13. The method for production according to claim 12, wherein X¹ and X⁴ in the sequence (1) are any of (A1) to (A8) below:
(A1) X¹ being glycine and X⁴ being threonine;
(A2) X¹ being glycine and X⁴ being serine;
(A3) X¹ being alanine and X⁴ being serine;
(A4) X¹ being alanine and X⁴ being threonine;
(A5) X¹ being alanine and X⁴ being alanine;
(A6) X¹ being glycine and X⁴ being alanine;
(A7) X¹ being phenylalanine and X⁴ being threonine; and
(A8) X¹ being phenylalanine and X⁴ being serine.

14. The method for production according to claim 12, wherein in the sequence (1), X² is serine, alanine, or aspartic acid.

15. The method for production according to claim 12, wherein X¹, X², X³, and X⁴ in the sequence (1) are any of (B1) to (B11) below:
(B1) X¹ being glycine, X² being serine, X³ being tyrosine, and X⁴ being threonine;
(B2) X¹ being glycine, X² being alanine, X³ being tyrosine, and X⁴ being threonine;
(B3) X¹ being glycine, X² being serine, X³ being tyrosine, and X⁴ being serine;
(B4) X¹ being glycine, X² being aspartic acid, X³ being tyrosine, and X⁴ being threonine;
(B5) X¹ being alanine, X² being serine, X³ being tyrosine, and X⁴ being serine;
(B6) X¹ being alanine, X² being serine, X³ being tyrosine, and X⁴ being threonine;
(B7) X¹ being alanine, X² being serine, X³ being tyrosine, and X⁴ being alanine;
(B8) X¹ being glycine, X² being serine, X³ being tyrosine, and X⁴ being alanine;
(B9) X¹ being phenylalanine, X² being serine, X³ being tyrosine, and X⁴ being threonine;
(B10) X¹ being phenylalanine, X² being serine, X³ being tyrosine, and X⁴ being serine; and
(B11) X¹ being glycine, X² being serine, X³ being tryptophan, and X⁴ being serine.

16. The method for production according to claim 12, wherein the histidine (His) residue at the 7-th position in the amino acid sequence represented by the sequence (1) is located between positions 120 and 160 from the N-terminus of the polypeptide employed as the phosphorylating enzyme.

17. The method for production according to claim 10, wherein the phosphorylating enzyme further comprises at least one of the amino acid sequences as defined in (iii) to (vi) below:
(iii) a polypeptide that has, on the N-terminal side of the amino acid sequence set forth in SEQ ID NO: 2, the amino acid sequence set forth in SEQ ID NO: 40 and has an activity to catalyze the glycerol phosphorylating reaction;
(iv) a polypeptide that has, on the N-terminal side of the amino acid sequence set forth in SEQ ID NO: 2, an amino acid sequence having one or a few amino acids substituted, deleted, inserted, or added in the amino acid sequence set forth in SEQ ID NO: 40 and has an activity to catalyze the glycerol phosphorylating reaction which is comparable or superior to that of the polypeptide as defined in (iii);
(v) a polypeptide that has, on the C-terminal side of the amino acid sequence set forth in SEQ ID NO: 2, the amino acid sequence set forth in SEQ ID NO: 41 and has an activity to catalyze the glycerol phosphorylating reaction; and
(vi) a polypeptide that has, on the C-terminal side of the amino acid sequence set forth in SEQ ID NO: 2, an amino acid sequence having one or a few amino acids substituted, deleted, inserted, or added in the amino acid sequence set forth in SEQ ID NO: 41 and has an activity to catalyze the glycerol phosphorylating reaction which is comparable or superior to that of the polypeptide as defined in (v).

18. The method for production according to any of claims 10 to 17, wherein the phosphorylating enzyme comprises a polypeptide as defined in (vii) or (viii) below:
(vii) a polypeptide that has the amino acid sequence set forth in any of SEQ ID NOs: 9, 15 to 17, 19, 22, and 25; or
(viii) a polypeptide that comprises an amino acid sequence having, in the amino acid sequence set forth in any of SEQ ID NOs: 9, 15 to 17, 19, 22, and 25, one or several amino acids substituted, deleted, inserted, or added in a region outside the amino acid sequence set forth in SEQ ID NO: 2 and has an activity to catalyze the glycerol phosphorylating reaction which is comparable or superior to that of the polypeptide as defined in (vii).

19. The method for production according to claim 1, wherein the phosphate group donor is a polyphosphoric acid.

20. The method for production according to claim 1, wherein the phosphorylated glycerol is α-glycerophosphate.

21. The method for production according to claim 1, wherein the pH of the reaction solution is from 4 to 5 in the step of allowing the phosphorylating enzyme to act on the glycerol in the presence of the phosphate group donor.

22. The method for production according to claim 1, wherein the glycerol is added in an amount of 1000 to 50000 parts by weight per part by weight of the phosphorylating enzyme, in the step of allowing the phosphorylating enzyme to act on the glycerol in the presence of the phosphate group donor.

23. The method for production according to claim 1, wherein the concentration of the phosphate group donor in the reaction solution is from 2% to 10% by weight in the step of allowing the phosphorylating enzyme to act on the glycerol in the presence of the phosphate group donor.


SEQUENCE LISTING (139 sequences)

SEQ ID NO: 1 - protein, 8 aa, artificial sequence
  amino acid sequence for active site.
XXXPSGHX

SEQ ID NO: 2 - protein, 18 aa, Gluconobacter oxydans
PDERLVLAPPPAPGSAAQ

SEQ ID NO: 3 - protein, 238 aa, Gluconacetobacter hansenii
MDAGYLTPATQPDATQYLPPPPQAGSARQAADDHAFESTRGLKGGARWALATSDADLRIEALLRSFSCAAGFTIDASKAPRLAALIHRMDVSEIPDMRNAKASWHRARPFVGNTQSICTEDDRSHLATSGAYPSGHTLLGWSTALVLAELLPDRSTEILQRGRVFGESRIVCGVHWASDVLEGYMTGAGDIAAMHGNPAFRADLDAARTELEGLRHEAPKPNPQACTIEHDAAAHSPL

SEQ ID NO: 4 - protein, 243 aa, Xanthomonas oryzae
MDAHGYLEKSELPDSLQLVPPPPQDDSAALANDETVSKAMLALRGTPRWELAAQDAVLRFPAAATHFSCALGIQIDQTSTPHLVRVLERSMRDASTATSAAKARYQRPRPFMRNAQPMCTPDDDAALRKNGSYPSGHTAIGWTWGLILSEIAPAHRDALLARGRAFGDSRLVCNVHWQSDVIQGRMVGAAAVAALHGNPAFEKDLAAARREIEKAQAKQPTAAAAAACNAEREALKTVLPGVM

SEQ ID NO: 5 - protein, 233 aa, Brevundimonas diminuta
MDHPHGYLTAENTPNAANFLPPPPAEGSLREQADIAAYRAMRSLEGSERWAIARADNEIETPGAPRAFDCALGFKFEPEQMPTLTLLMGKMLGDLEMIQTPAKKGYFRKRPFVVEPLPTCIAPETWLAASGSYPSGHSALGWAWGLVLAELAPDRADAILRRGLAYGESRAVCGVHYPSDVEAGRIVGATIVTRLKADPAFQADFAKAKEEFDAARAAATEATAACPASLARQ

SEQ ID NO: 6 - protein, 232 aa, Sphingomonas trueperi
MDTAPYLAAGQYPDGMAILPPPPALDSPGAALDMAVFRATRKLEGTPRWRIATDDVTNDPLRRNACAMGMVLDVKTAPALARLLDRAGTGPVVGRVKAAYQVPRPYLREDGPICEAKTAHLASNGDYPSGHTANGWLEAQILAEVMPDKATAILARGRAYGESRAICGSHSKSAVEAGYMAGASVFAVLQTSPAYQRDLAAARQEAARLRTTAPRPDAQSCVAEAEALRVRP

SEQ ID NO: 7 - protein, 245 aa, Zymomonas mobilis
MDLSQSVSAHTEKSEPSSTYHFHSDPLLYLAPPPTSGSPLQAHDDQTFNSTRQLKGSTRWALATQDADLHLASVLKDYACAAGMNLDIAQLPHLANLIKRALRTEYDDIGRAKNNWNRKRPFVDTDQPICTEKDREGLGKQGSYPSGHTTIGWSVALILAELIPDHAANILQRGQIFGTSRIVCGAHWFSDVQAGYIMASGEIAALHGDADFRRDMELARKELEKARTSAHTPDDLLCKIEQSAR

SEQ ID NO: 8 - protein, 223 aa, Desulfovibrio magneticus
MDIGINSDPQLAWSETQFVSPQQVDLARLLPPPPAMDSAEQRDEIALLLQLQKDRTPDMVAFAQADAAREVFRFTDVVGPQFTAEKLPVAAAFFKAVKENGDAILGNAKKHWDRPRPYAASSQIDPCVPKPGNASYPSGHSTYGTLMGIILANMVPEKAQALAARAEQYRFNREIGGVHYPSDVAAGRITGTVIAAFLFNSPEFQQQYAAARAEVRSALGLAQ

SEQ ID NO: 9 - protein, 244 aa, Gluconobacter oxydans
MDTSATAQGGILPDSAAPDERLVLAPPPAPGSAAQSDDDRVFHVTRALKDTPRWKLAQSDADLDPAHVVRDFSCAAGFEIDLARAPHLARVLERIRHAVGHRTSDVKKYWHRTRPFVGTNLPICTSPEGLGLNASYPSGHTTAGYGMALLLAHLMPEHASAILQRGRVFGESRIVCGAHWKSDVQAGYLNASSLMDVLLARPELQDDLAAARQELLAMQGTAPVPDAGTCAVEHDAAIHSLLSE

SEQ ID NO: 10 - protein, 217 aa, Flavobacterium johnsoniae
MDALLDGYLSEAEMPDSLLLLSPPPEHNSSLFDLDLEHAKKAVESKDKERFLQAARDADLSFPFAVKSFEPILGIEISETKTPKFYVLMRRVMTDAGLSTYAAKNHYKRERPFMVNNQKTCTPDQENILYKVGSFPSGHAAVGWAWSLVLIKLFPDKQEEILKRGHDFGESRVICNAHWYSDVEMGRVMGRAAVECLCVNSAFLSDLEEVKKEMAGA

SEQ ID NO: 11 - protein, 253 aa, Methylobacterium ectorquens
MDSAPSLEKDKLAAAAPKGYLSEEATPNLVAILPPPPAGHSAAEAADRAVYNAARAFQGSPRWALATDDVADGGAALLQDYACVLGQRIDQASVPDLMRLLDRARIDIARATRVAKRRYRRLRPFVGNDLPICVARTAELADSFSYPSGHASQGWAYGLIMANLMPEKATQFLVRSRLYGESRVVCGVHWLSDIEAARTGASALVAVLLADPGFRTDLERARTDLKRALSGEGAKPDPALCAREDAAARQPLL

SEQ ID NO: 12 - protein, 242 aa, Acetobacter pasteurianus
MDSSLFGYTAQAQQFTLPDGRAFLPPPPQAEEPAQQADLRAFEKTRGLKDKARWKLAQNDANLNPSHVIKDFSCAAGFNLDPEKLPAMVNLLTSLAQPVEQDVSNEKDFWKRRRPFVGTNKDICTAHSDGLDNSYAYPSGHTTWGWLTASILASALPDRATQIMQRGRIFGESRIVCGVHWKSDVQAGYMNGSAIFAALQEQPTFTEQMAKVRQELLALRDAKTAPDAKTCAVEQQAAQDAF

SEQ ID NO: 13 - protein, 253 aa, artificial sequence
  variant a
MDSAPSLEKDKLAAAAPKGYLSEEATPNLVAILPPPPAGHSAAEAADRAVYNAARAFQGSPRWALATDDVADGGAALLQDYACVLGQRIDQASVPDLMRLLDRARIDIARATRVAKRRYRRLRPFVGNDLPICVARTAELADSGSYPSGHTSQGWAYGLIMANLMPEKATQFLVRSRLYGESRVVCGVHWLSDIEAARTGASALVAVLLADPGFRTDLERARTDLKRALSGEGAKPDPALCAREDAAARQPLL

SEQ ID NO: 14 - protein, 245 aa, artificial sequence
  variant A
MDSSLFGYTAQAQQFTLPDGRAFLPPPPQAEEPAQQADLRAFEKTRGLKDKARWKLAQNDANLNPSHVIKDFSCAAGFNLDPEKLPAMVNLLTSLAQPVEQDVSNEKDFWKRRRPFVGTNKDICTAHSDGLDNSYAYPSGHTTAGYGMALLLAHLMPEHASAILQRGRVFGESRIVCGAHWKSDVQAGYLNASSLMDVLLARPELQDDLAAARQELLAMQGTAPVPDAGTCAVEHDAAIHSLLSE

SEQ ID NO: 15 - protein, 241 aa, artificial sequence
  variant B
MDTSATAQGGILPDSAAPDERLVLAPPPAPGSAAQSDDDRVFHVTRALKDTPRWKLAQSDADLDPAHVVRDFSCAAGFEIDLARAPHLARVLERIRHAVGHRTSDVKKYWHRTRPFVGTNLPICTSPEGLGLNASYPSGHTTAGWLTASILASALPDRATQIMQRGRIFGESRIVCGVHWKSDVQAGYMNGSAIFAALQEQPTFTEQMAKVRQELLALRDAKTAPDAKTCAVEQQAAQDAF

SEQ ID NO: 16 - protein, 242 aa, artificial sequence
  variant C
MDTSATAQGGILPDSAAPDERLVLAPPPAPGSAAQSDDDRVFHVTRALKDTPRWKLAQSDADLDPAHVVRDFSCAAGFEIDLARAPHLARVLERIRHAVGHRTSDVKKYWHRTRPFVGTNLPICTAHSDGLDNSYAYPSGHTTWGWLTASILASALPDRATQIMQRGRIFGESRIVCGVHWKSDVQAGYMNGSAIFAALQEQPTFTEQMAKVRQELLALRDAKTAPDAKTCAVEQQAAQDAF

SEQ ID NO: 17 - protein, 242 aa, artificial sequence
  variant D
MDTSATAQGGILPDSAAPDERLVLAPPPAPGSAAQSDDDRVFHVTRALKDTPRWKLAQSDADLDPAHVVRDFSCAAGFNLDPEKLPAMVNLLTSLAQPVEQDVSNEKDFWKRRRPFVGTNKDICTAHSDGLDNSYAYPSGHTTWGWLTASILASALPDRATQIMQRGRIFGESRIVCGVHWKSDVQAGYMNGSAIFAALQEQPTFTEQMAKVRQELLALRDAKTAPDAKTCAVEQQAAQDAF

SEQ ID NO: 18 - protein, 241 aa, artificial sequence
  variant E
MDSSLFGYTAQAQQFTLPDGRAFLPPPPQAEEPAQQADLRAFEKTRGLKDKARWKLAQNDANLNPSHVIKDFSCAAGFNLDPEKLPAMVNLLTSLAQPVEQDVSNEKDFWKRRRPFVGTNKDICTSPEGLGLNASYPSGHTTAGWLTASILASALPDRATQIMQRGRIFGESRIVCGVHWKSDVQAGYMNGSAIFAALQEQPTFTEQMAKVRQELLALRDAKTAPDAKTCAVEQQAAQDAF

SEQ ID NO: 19 - protein, 242 aa, artificial sequence
  variant F
MDSSLFGYTAQAQQFTLPDERLVLAPPPAPGSAAQSDDDRVFHVTRALKDTPRWKLAQSDADLDPAHVVRDFSCAAGFNLDPEKLPAMVNLLTSLAQPVEQDVSNEKDFWKRRRPFVGTNKDICTAHSDGLDNSYAYPSGHTTWGWLTASILASALPDRATQIMQRGRIFGESRIVCGVHWKSDVQAGYMNGSAIFAALQEQPTFTEQMAKVRQELLALRDAKTAPDAKTCAVEQQAAQDAF

SEQ ID NO: 20 - protein, 242 aa, artificial sequence
  variant G
MDSSLFGYTAQAQQFTLPDGRAFLPPPPAPGSAAQSDDDRVFHVTRALKDTPRWKLAQSDADLDPAHVVRDFSCAAGFNLDPEKLPAMVNLLTSLAQPVEQDVSNEKDFWKRRRPFVGTNKDICTAHSDGLDNSYAYPSGHTTWGWLTASILASALPDRATQIMQRGRIFGESRIVCGVHWKSDVQAGYMNGSAIFAALQEQPTFTEQMAKVRQELLALRDAKTAPDAKTCAVEQQAAQDAF

SEQ ID NO: 21 - protein, 242 aa, artificial sequence
  variant H
MDSSLFGYTAQAQQFTLPDGRAFLPPPPQAEEPAQQADLRAFEKTRGLKDKARWKLAQSDADLDPAHVVRDFSCAAGFNLDPEKLPAMVNLLTSLAQPVEQDVSNEKDFWKRRRPFVGTNKDICTAHSDGLDNSYAYPSGHTTWGWLTASILASALPDRATQIMQRGRIFGESRIVCGVHWKSDVQAGYMNGSAIFAALQEQPTFTEQMAKVRQELLALRDAKTAPDAKTCAVEQQAAQDAF

SEQ ID NO: 22 - protein, 242 aa, artificial sequence
  variant I
MDTSATAQGGILPDSAAPDERLVLAPPPAPGSAAQSDDDRVFHVTRALKDTPRWKLAQNDANLNPSHVIKDFSCAAGFNLDPEKLPAMVNLLTSLAQPVEQDVSNEKDFWKRRRPFVGTNKDICTAHSDGLDNSYAYPSGHTTWGWLTASILASALPDRATQIMQRGRIFGESRIVCGVHWKSDVQAGYMNGSAIFAALQEQPTFTEQMAKVRQELLALRDAKTAPDAKTCAVEQQAAQDAF

SEQ ID NO: 23 - protein, 242 aa, artificial sequence
  variant J
MDTSATAQGGILPDSAAPDERLVLAPPPQAEEPAQQADLRAFEKTRGLKDKARWKLAQNDANLNPSHVIKDFSCAAGFNLDPEKLPAMVNLLTSLAQPVEQDVSNEKDFWKRRRPFVGTNKDICTAHSDGLDNSYAYPSGHTTWGWLTASILASALPDRATQIMQRGRIFGESRIVCGVHWKSDVQAGYMNGSAIFAALQEQPTFTEQMAKVRQELLALRDAKTAPDAKTCAVEQQAAQDAF

SEQ ID NO: 24 - protein, 242 aa, artificial sequence
  variant K
MDTSATAQGGILPDSAAPDGRAFLPPPPQAEEPAQQADLRAFEKTRGLKDKARWKLAQNDANLNPSHVIKDFSCAAGFNLDPEKLPAMVNLLTSLAQPVEQDVSNEKDFWKRRRPFVGTNKDICTAHSDGLDNSYAYPSGHTTWGWLTASILASALPDRATQIMQRGRIFGESRIVCGVHWKSDVQAGYMNGSAIFAALQEQPTFTEQMAKVRQELLALRDAKTAPDAKTCAVEQQAAQDAF

SEQ ID NO: 25 - protein, 242 aa, artificial sequence
  variant L
MDTSATAQGGILPDSAAPDERLVLAPPPAPGSAAQQADLRAFEKTRGLKDKARWKLAQNDANLNPSHVIKDFSCAAGFNLDPEKLPAMVNLLTSLAQPVEQDVSNEKDFWKRRRPFVGTNKDICTAHSDGLDNSYAYPSGHTTWGWLTASILASALPDRATQIMQRGRIFGESRIVCGVHWKSDVQAGYMNGSAIFAALQEQPTFTEQMAKVRQELLALRDAKTAPDAKTCAVEQQAAQDAF

SEQ ID NO: 26 - protein, 129 aa, Gluconacetobacter hansenii
MDAGYLTPATQPDATQYLPPPPQAGSARQAADDHAFESTRGLKGGARWALATSDADLRIEALLRSFSCAAGFTIDASKAPRLAALIHRMDVSEIPDMRNAKASWHRARPFVGNTQSICTEDDRSHLATS

SEQ ID NO: 27 - protein, 130 aa, Xanthomonas oryzae
MDAHGYLEKSELPDSLQLVPPPPQDDSAALANDETVSKAMLALRGTPRWELAAQDAVLRFPAAATHFSCALGIQIDQTSTPHLVRVLERSMRDASTATSAAKARYQRPRPFMRNAQPMCTPDDDAALRKN

SEQ ID NO: 28 - protein, 130 aa, Brevundimonas diminuta
MDHPHGYLTAENTPNAANFLPPPPAEGSLREQADIAAYRAMRSLEGSERWAIARADNEIETPGAPRAFDCALGFKFEPEQMPTLTLLMGKMLGDLEMIQTPAKKGYFRKRPFVVEPLPTCIAPETWLAAS

SEQ ID NO: 29 - protein, 124 aa, Sphingomonas trueperi
MDTAPYLAAGQYPDGMAILPPPPALDSPGAALDMAVFRATRKLEGTPRWRIATDDVTNDPLRRNACAMGMVLDVKTAPALARLLDRAGTGPVVGRVKAAYQVPRPYLREDGPICEAKTAHLASN

SEQ ID NO: 30 - protein, 141 aa, Zymomonas mobilis
MDLSQSVSAHTEKSEPSSTYHFHSDPLLYLAPPPTSGSPLQAHDDQTFNSTRQLKGSTRWALATQDADLHLASVLKDYACAAGMNLDIAQLPHLANLIKRALRTEYDDIGRAKNNWNRKRPFVDTDQPICTEKDREGLGKQ

SEQ ID NO: 31 - protein, 133 aa, Desulfovibrio magneticus
MDIGINSDPQLAWSETQFVSPQQVDLARLLPPPPAMDSAEQRDEIALLLQLQKDRTPDMVAFAQADAAREVFRFTDVVGPQFTAEKLPVAAAFFKAVKENGDAILGNAKKHWDRPRPYAASSQIDPCVPKPGN

SEQ ID NO: 32 - protein, 133 aa, Gluconobacter oxydans
MDTSATAQGGILPDSAAPDERLVLAPPPAPGSAAQSDDDRVFHVTRALKDTPRWKLAQSDADLDPAHVVRDFSCAAGFEIDLARAPHLARVLERIRHAVGHRTSDVKKYWHRTRPFVGTNLPICTSPEGLGLN

SEQ ID NO: 33 - protein, 101 aa, Gluconacetobacter hansenii
LLGWSTALVLAELLPDRSTEILQRGRVFGESRIVCGVHWASDVLEGYMTGAGDIAAMHGNPAFRADLDAARTELEGLRHEAPKPNPQACTIEHDAAAHSPL

SEQ ID NO: 34 - protein, 105 aa, Xanthomonas oryzae
AIGWTWGLILSEIAPAHRDALLARGRAFGDSRLVCNVHWQSDVIQGRMVGAAAVAALHGNPAFEKDLAAARREIEKAQAKQPTAAAAAACNAEREALKTVLPGVM

SEQ ID NO: 35 - protein, 95 aa, Brevundimonas diminuta
ALGWAWGLVLAELAPDRADAILRRGLAYGESRAVCGVHYPSDVEAGRIVGATIVTRLKADPAFQADFAKAKEEFDAARAAATEATAACPASLARQ

SEQ ID NO: 36 - protein, 100 aa, Sphingomonas trueperi
ANGWLEAQILAEVMPDKATAILARGRAYGESRAICGSHSKSAVEAGYMAGASVFAVLQTSPAYQRDLAAARQEAARLRTTAPRPDAQSCVAEAEALRVRP

SEQ ID NO: 37 - protein, 96 aa, Zymomonas mobilis
TIGWSVALILAELIPDHAANILQRGQIFGTSRIVCGAHWFSDVQAGYIMASGEIAALHGDADFRRDMELARKELEKARTSAHTPDDLLCKIEQSAR

SEQ ID NO: 38 - protein, 82 aa, Desulfovibrio magneticus
TYGTLMGIILANMVPEKAQALAARAEQYRFNREIGGVHYPSDVAAGRITGTVIAAFLFNSPEFQQQYAAARAEVRSALGLAQ

SEQ ID NO: 39 - protein, 103 aa, Gluconobacter oxydans
TAGYGMALLLAHLMPEHASAILQRGRVFGESRIVCGAHWKSDVQAGYLNASSLMDVLLARPELQDDLAAARQELLAMQGTAPVPDAGTCAVEHDAAIHSLLSE

SEQ ID NO: 40 - protein, 17 aa, Gluconacetobacter hansenii
MDTSATAQGGILPDSAA

SEQ ID NO: 41 - protein, 209 aa, Gluconacetobacter hansenii
SDDDRVFHVTRALKDTPRWKLAQSDADLDPAHVVRDFSCAAGFEIDLARAPHLARVLERIRHAVGHRTSDVKKYWHRTRPFVGTNLPICTSPEGLGLNASYPSGHTTAGYGMALLLAHLMPEHASAILQRGRVFGESRIVCGAHWKSDVQAGYLNASSLMDVLLARPELQDDLAAARQELLAMQGTAPVPDAGTCAVEHDAAIHSLLSE

SEQ ID NO: 42 - DNA, 36 bp, artificial sequence
  Primer 45
tgcacccatggacgcagggtatctaacacctgcgac

SEQ ID NO: 43 - DNA, 37 bp, artificial sequence
  primer 46
caataagcttgagcggagaatgggccgcagcatcatg

SEQ ID NO: 44 - DNA, 34 bp, artificial sequence
  primer 47
gatcgccatggacgcgcatggatacctggaaaag

SEQ ID NO: 45 - DNA, 35 bp, artificial sequence
  primer 48
ttccaagcttcatcacaccaggcaacaccgtcttc

SEQ ID NO: 46 - DNA, 37 bp, artificial sequence
  primer 49
agcttccatggatcatccgcacggctatctgaccgcc

SEQ ID NO: 47 - DNA, 36 bp, artificial sequence
  primer 50
atcaaagcttctgacgcgccagcgaggcggggcagg

SEQ ID NO: 48 - DNA, 32 bp, artificial sequence
  primer 51
tcaggccatggacacggcgccgtacctcgccg

SEQ ID NO: 49 - DNA, 34 bp, artificial sequence
  primer 52
aagaaagcttggggcggacgcgaagggcctccgc

SEQ ID NO: 50 - DNA, 34 bp, artificial sequence
  primer 53
tctggccatggatctttctcaaagcgtttcagct

SEQ ID NO: 51 - DNA, 33 bp, artificial sequence
  primer 54
tatgaagcttgcgagcgctttgttcaatcttgc

SEQ ID NO: 52 - DNA, 37 bp, artificial sequence
  primer 55
tatggatccgcagctggcctggtccgaaacgcagttc

SEQ ID NO: 53 - DNA, 39 bp, artificial sequence
  primer 56
cctcaagcttctgggccagacccagggcgctgcggactt

SEQ ID NO: 54 - DNA, 38 bp, artificial sequence
  primer 57
taacgcccatggatacgtccgctaccgcccaaggcggc

SEQ ID NO: 55 - DNA, 37 bp, artificial sequence
  primner 58
aacaaagcttttcgctgagcagggaatggatcgcggc

SEQ ID NO: 56 - DNA, 35 bp, artificial sequence
  primer 59
cttagaccatggatgcattgctggatggatatttg

SEQ ID NO: 57 - DNA, 36 bp, artificial sequence
  primer 60
ccataagcttagctcctgccatttcttttttaacct

SEQ ID NO: 58 - DNA, 37 bp, artificial sequence
  primer 61
tacgttccatggattccgccccctcgctcgagaagga

SEQ ID NO: 59 - DNA, 36 bp, artificial seuqence
  primer 62
atccaagcttgagcaggggctggcgcgcggccgcat

SEQ ID NO: 60 - DNA, 35 bp, artificial sequence
  primer 63
cttagaccatggatagcagcctttttggatatacc

SEQ ID NO: 61 - DNA, 37 bp, artificial sequence
  primer 64
aagtaagcttaaaagcgtcttgcgcagcctgctgttc

SEQ ID NO: 62 - DNA, 37 bp, artificial sequence; identical to SEQ ID NO: 58
  primer (3)

SEQ ID NO: 63 - DNA, 36 bp, artificial sequence; identical to SEQ ID NO: 59
  primer (4)

SEQ ID NO: 64 - DNA, 36 bp, artificial sequence
  primer (11)
agcggatcctatccctccggtcatactagccagggg

SEQ ID NO: 65 - DNA, 33 bp, artificial sequence
  primer (12)
ataggatccgctgtccgcgagttcggccgtgcg

SEQ ID NO: 66 - DNA, 35 bp, artificial sequence; identical to SEQ ID NO: 60
  primer 1

SEQ ID NO: 67 - DNA, 37 bp, artificial sequence; identical to SEQ ID NO: 61
  primer 2

SEQ ID NO: 68 - DNA, 38 bp, artificial sequence; identical to SEQ ID NO: 54
  primer 3

SEQ ID NO: 69 - DNA, 37 bp, artificial sequence; identical to SEQ ID NO: 55
  primer 4

SEQ ID NO: 70 - DNA, 31 bp, artificial sequence
  primer 5
gactgcaggctacgggatggcgctgctcctg

SEQ ID NO: 71 - DNA, 28 bp, artificial sequence
  primer 6
atcctgcagttgtatggccagagggata

SEQ ID NO: 72 - DNA, 29 bp, artificial sequence
  primer 7
caactgcaggatggctcacggcatccatt

SEQ ID NO: 73 - DNA, 33 bp, artificial sequence
  primer 8
tcctgcagtcgtatgacccgacggataggacgc

SEQ ID NO: 74 - DNA, 36 bp, artificial sequence
  primer 9
aataaacacatatgcacagcacattcagatgggctt

SEQ ID NO: 75 - DNA, 36 bp, artificial sequence
  primer 10
gagagctgcatatgggcaggttcgtacccacgaacg

SEQ ID NO: 76 - DNA, 33 bp, artificial sequence
  primer 11
tttaatgtcgacccagaaaaacttcccgcaatg

SEQ ID NO: 77 - DNA, 35 bp, artificial sequence
  primer 12
gccagctcgagattgaaacccgccgcacaggagaa

SEQ ID NO: 78 - DNA, 44 bp, artificial sequence
  primer 13
acactagtccggaaggtcttggtctcaacgcttcctatccctct

SEQ ID NO: 79 - DNA, 32 bp, artificial sequence
  primer 14
ggactagtgcagatgtctttatttgtgccgac

SEQ ID NO: 80 - DNA, 27 bp, artificial sequence
  primer 15
cgactagttctggctccgccgccggcg

SEQ ID NO: 81 - DNA, 31 bp, artificial sequence
  primer 16
aaactagtcgttcatcaggtagggtaaactg

SEQ ID NO: 82 - DNA, 33 bp, artificial sequence
  primer 17
ggaactgcagcgcagagtgatgacgaccgggtg

SEQ ID NO: 83 - DNA, 37 bp, artificial sequence
  primer 18
agtgctgcagatccgggcgctggtggcgggggcaaaa

SEQ ID NO: 84 - DNA, 33 bp, artificial sequence
  primer 19
cgctggaagctagcgcagagcgatgcggatctg

SEQ ID NO: 85 - DNA, 37 bp, artificial sequence
  primer 20
attctgtagctagcttccagcgcgccttatcttttaa

SEQ ID NO: 86 - DNA, 33 bp, artificial sequence
  primer 21
cgctggaagctagcccagaatgacgccaacctt

SEQ ID NO: 87 - DNA, 36 bp, artificial sequence
  primer 22
gctctgagctagcttccagcgcggcgtgtccttcag

SEQ ID NO: 88 - DNA, 38 bp, artificial sequence
  primer 23
gacttcagctagcaccgccaccacaggcagaagaaccc

SEQ ID NO: 89 - DNA, 36 bp, artificial sequence
  primer 24
cggcggagctagcacgagacgctcgtccggagccgc

SEQ ID NO: 90 - DNA, 33 bp, artificial sequence
  primer 25
cagtctgcagcacctgatggacgtgcatttttg

SEQ ID NO: 91 - DNA, 32 bp, artificial sequence
  primer 26
ggagctgcagagtccggcaggatgccgccttg

SEQ ID NO: 92 - DNA, 32 bp, artificial sequence
  primer 27
aacctgcagcacaacaggcagacttgcgggct

SEQ ID NO: 93 - DNA, 31 bp, artificial sequence
  primer 28
ctgctgcagatccgggcgccggcggcggagc

SEQ ID NO: 94 - DNA, 714 bp, Gluconacetobacter hansenii
atggacgcagggtatctaacacctgcgacgcagcctgatgccacacagtaccttcccccgccgccgcaggctggctcggcgcgtcaggccgcggatgaccatgcctttgaatccacgcgtgggctgaagggcggcgcgcgctgggcactggcgacatccgatgcggacctgcgtatcgaggcgctgctgcgcagcttttcctgtgcggcaggctttacgatcgacgccagcaaggcaccgcgccttgccgcgctgatccatcggatggatgtcagcgaaattcccgatatgcgcaacgccaaggcatcatggcatcgcgcacgtcctttcgtgggcaatacgcagtcgatctgcacagaggacgatcggtcccacctggcgacatcgggggcttatccttccgggcacacccttctgggctggagtacggcgctggtgctggcggaacttctgccggaccgttcgaccgaaatcctgcaacgtgggcgcgtattcggtgaaagccgtatcgtctgtggcgtgcattgggccagtgacgtgctggagggatatatgaccggggcaggcgatattgccgccatgcacggcaaccctgccttccgggcggatctggatgccgcacggaccgagttggagggcctgcgtcacgaagcccccaaacccaatccgcaggcttgtacgatcgagcatgatgctgcggcccattctccgctc

SEQ ID NO: 95 - DNA, 729 bp, Xanthomonas oryzae
atggacgcgcatggatacctggaaaagagcgaactgcccgacagcctgcagctggtgccgccgccaccacaggatgacagcgccgcgctcgccaacgacgagacggtttccaaggccatgctcgccctgcgcggcacgccgcgctgggagctggccgcgcaggacgcagtgctgcgctttccggccgcagccacgcatttcagctgcgcgctcggcatacagatcgaccagaccagcaccccgcatctggtgcgcgtgctcgaacgcagcatgcgcgatgccagtaccgccaccagtgccgccaaggcgcgctatcagcgtccacgcccgttcatgcgcaatgcgcagccgatgtgcaccccggacgatgacgccgccctgcgcaagaacggctcttacccttccggtcacaccgccatcggctggacctggggcctgatcctgtcggaaatcgcacccgcccaccgcgatgccctgctcgcccgcggccgtgccttcggcgatagccgcctggtctgcaacgtgcactggcagagcgatgtcatccaaggccgcatggtgggcgccgccgccgtcgccgccttgcacggcaaccccgcattcgaaaaagacctggccgccgcacgccgcgagatcgagaaagcgcaggcgaagcagccgacggcagcggcagcggcggcgtgtaatgcggaacgcgaagcgctgaagacggtgttgcctggtgtgatg

SEQ ID NO: 96 - DNA, 699 bp, Brevundimonas diminuta
atggatcatccgcacggctatctgaccgccgagaacacgcccaacgccgccaacttcctgccgccgccgcccgccgaaggctcgttgcgcgaacaggccgacatcgccgcctatcgcgccatgcggtcgctggaaggctccgagcgctgggccatcgcccgcgccgacaatgagatcgaaacgcccggcgccccgcgcgccttcgactgcgccctcggcttcaagttcgagccggaacagatgccgaccctgacgctgttgatgggcaagatgctgggcgatctggagatgatccaaacgcccgccaagaagggctatttccgcaaacgccccttcgtggtcgagcccctgccgacctgcatcgcgcccgagacctggctggcggccagcggctcctatccgtcgggccactcggcgctgggctgggcctggggcctggtgctggccgaactggcgcccgaccgcgccgacgccatcctgcgccgcggcctggcctatggcgaaagccgcgcggtgtgcggcgtccactatcccagcgacgtcgaggcgggccgcatcgtcggcgcgaccatcgtcacccggctgaaggccgatccggcgttccaggccgatttcgccaaggccaaggaagagttcgacgccgcccgcgccgccgcgaccgaggctacggccgcctgccccgcctcgctggcgcgtcag

SEQ ID NO: 97 - DNA, 696 bp, Sphingomonas trueperi
atggacacggcgccgtacctcgccgcgggccaatatcccgacggcatggcgatcctgccgccgccgccggcgctcgacagccccggcgctgccctggacatggcggtgttccgggcaacgcggaagctggagggcaccccgcgctggcggatcgccaccgacgacgtcaccaacgatccgctgcgccgcaacgcctgcgcgatggggatggtgctcgacgtcaagactgcgccggcacttgcccggctgctcgatcgcgcaggcacgggcccggtcgtcggcagggtgaaggctgcctaccaagtgccgcgtccctatctgcgcgaggacggcccgatctgcgaggccaagaccgcacatctcgccagcaatggcgactacccgtcggggcacaccgccaatggttggctggaagcgcagatcctcgccgaggtgatgcccgacaaggcgactgcgatcctcgcgcgcggccgtgcctatggcgagagccgggcgatttgcgggtcgcacagcaagagcgcggtcgaagccggctacatggccggcgcctcggtgttcgcggtgctgcagacctcgcccgcctaccagcgggatctcgcggcggcgcggcaggaagcggcgcggctgcgcactaccgcgccgcgccccgatgcacaaagctgtgtcgcggaagcggaggcccttcgcgtccgcccc

SEQ ID NO: 98 - DNA, 735 bp, Zymomonas mobilis
atggatctttctcaaagcgtttcagctcatacagaaaaaagtgaaccctcctcgacttatcatttccacagcgatccccttctttaccttgcgcccccacccacttccggcagtccattacaggcgcatgatgatcaaacctttaacagcaccagacaattaaaaggtagcacgcgctgggcattggcaactcaagatgccgatcttcatctcgcttcagttctcaaagactatgcctgcgccgcaggaatgaatctcgatattgcgcaattaccgcatcttgccaatttgattaaacgcgcacttcgcaccgaatatgacgatattggcagagccaaaaataactggaatcgcaaacggccttttgtggataccgatcaacccatctgcacggaaaaagatcgcgaaggtctgggaaaacaaggctcctatccttcaggtcatacgactatcggttggagcgttgcactcattctggctgaattgatccccgatcatgcggcgaatattttgcagcgtggccaaatttttggaaccagccggattgtctgcggcgcccattggttcagcgatgtgcaggcaggctatatcatggcatcgggcgaaattgcagctttacatggggatgccgatttccgccgagatatggaattagctcggaaagaattagaaaaggcacgcacatcagcgcacacgccagacgatcttctatgcaagattgaacaaagcgctcgc

SEQ ID NO: 99 - DNA, 645 bp, Desulfovibrio magneticus
ccgcagctggcctggtccgaaacgcagttcgtcagcccgcagcaagtcgatctggcccggctgctgccgccgccgccggccatggactcggccgagcagcgcgacgagatcgccctgctcctgcaactgcaaaaggaccgcaccccggacatggtggcctttgcccaggccgacgccgcccgcgaggttttccgcttcaccgatgtggtcggcccccagttcacggctgaaaagctacccgtggcggcggcctttttcaaggcggtcaaggaaaacggcgacgccattctcggcaacgccaagaagcactgggaccgcccccgaccctatgccgctagttcgcaaatcgatccctgcgtgcccaagcccggcaacgcctcctaccccagcggccattccacctacggcaccttgatgggcatcatcctggccaacatggtcccggaaaaggcccaagccctggcggcccgggccgagcagtaccgcttcaaccgggagatcggcggcgtccactatcccagcgacgtggccgccggccgcatcaccggcacggtcatcgccgccttcctgttcaacagcccggaattccagcagcagtacgccgccgcccgggccgaagtccgcagcgccctggggctggcccag

SEQ ID NO: 100 - DNA, 732 bp, Gluconobacter oxydans
atggatacgtccgctaccgcccaaggcggcatcctgccggacagtgcggctccggacgagcgtctcgttctggctccgccgccggcgcccggaagtgccgcgcagagtgatgacgaccgggtgtttcatgtcacgcgcgcgctgaaggacacgccgcgctggaaactggcgcagagcgatgcggatctggatccggcgcatgtggtccgggatttctcctgtgcggcgggtttcgagatcgatctggccagggcgccgcatctggcccgtgttctggagcgcatccgccatgccgtcgggcaccggacttcggacgtgaagaaatactggcaccgtacgcggccgttcgtgggtacgaacctgccgatctgcacgtctcccgaagggctggggctgaacgcgtcctatccgtcgggtcatacgacggcgggatacgggatggcgctgctcctggcgcatctgatgccggaacatgcttcggccattctccagcgcgggcgtgtgttcggggaaagccggatcgtctgcggagcgcactggaagtcggatgtccaggcgggatacctgaatgcgtcttcgctgatggacgtgctgctggcgcggccggagcttcaggacgatctggcggcggcgcggcaggagctgctggccatgcagggaacggccccggttcctgatgcggggacgtgtgccgtagagcacgacgccgcgatccattccctgctcagcgaa

SEQ ID NO: 101 - DNA, 651 bp, Flavobacterium johnsoniae
atggatgcattgctggatggatatttgtctgaggcagaaatgcctgatagtcttcttttattgtcgccgcctccggaacataactcgagcctttttgatcttgatttagagcatgcaaaaaaggctgttgagtctaaggataaagagcgttttttacaggctgcaagagatgcagatctgtcctttccttttgctgttaaatcatttgaaccaattttaggtatagaaattagtgaaacaaaaacacctaagttttatgttcttatgcgaagagtaatgaccgatgcgggattatctacttatgcggctaaaaatcattataaacgagaacggccgtttatggttaataatcaaaaaacctgcactccggatcaggaaaatatattatataaagttgggtcttttccttcaggtcatgcggctgtaggctgggcatggtctttggttttgattaaactttttccggataaacaggaagagattttaaaacgcggacatgactttggtgaaagccgtgttatttgtaatgcacattggtatagtgatgttgaaatgggaagagttatggggcgggctgctgttgagtgtctttgtgttaattctgcttttctatccgatttggaggaggttaaaaaagaaatggcaggagct

SEQ ID NO: 102 - DNA, 759 bp, Methylobacterium ectorquens
atggattccgccccctcgctcgagaaggacaagctcgcagccgcggctccgaagggctatctcagcgaggaggcgacgccgaacctcgtggcgatcctgccgccgccccccgcgggccactcggcggcggaggcggcggaccgggcggtctacaacgcgacccgcgccttccagggcagcccgcgctgggccctcgccaccgacgacgtcgccgatggcggtgccgcgcttctcgaggattatgcctgcgtcctcggccagcgcatcgatcaggcgaacgtgccggacctgatgcggctcctcgaccgggcccggatcgacatcgcccgcgccacccgcgtggccaagcggcgctaccgtcgcctgcgccccttcgtcggcaacgacttgccgatctgcgtcgcccgcacggccgaactcgcggacagcttcagctatccctccggtcatgccagccaggggtgggcctacgggctgatcatggcgaacctgatgccggagaaggcgacgcagttgctggtgcgaagccggctttatggcgagagccgggtcgtgtgtggggtccactggctcagcgatatcgaggccgcccgcaccggcgcctcggcgctggtcgccgtactgctcgccgatcccagcttccgcaccgatctggagcgcgcccgggccgacctcaaacgcgcgctgagcggggagggggcgaaacccgatcccgcactctgtgcccgcgaggatgcggccgcgcgccagcccctgctc

SEQ ID NO: 103 - DNA, 726 bp, Acetobacter pasteurianus
atggatagcagcctttttggatataccgcacaggcacaacagtttaccctacctgatggacgtgcatttttgcccccgccaccacaggcagaagaacccgcacaacaggcagacttgcgggcttttgaaaaaacacgcggattaaaagataaggcgcgctggaaattggcccagaatgacgccaaccttaacccatcccatgtcatcaaggatttttcatgtgcggccgggtttaatctagacccagaaaaacttcccgcaatggtcaatcttctcacatcgctcgcgcagccagtagagcaagatgtttcaaacgaaaaagacttctggaagcggcgcagaccttttgtcggcacaaataaagacatctgcacagcacattcagatgggcttgataacagctatgcctatccctctggccatacaacatggggatggctcacggcatccattcttgccagcgctctgccagatcgtgcaacacagattatgcagcgcggccgtatatttggtgaaagccgtattgtctgcggcgtgcactggaaaagcgacgtacaggctggttacatgaacggaagcgccatatttgcggcacttcaggaacaacccacatttacagagcaaatggcaaaagttcgtcaggaactacttgccctgcgtgatgcaaaaacagcaccagacgcaaaaacttgcgcggtagaacagcaggctgcgcaagacgctttt

SEQ ID NO: 104 - protein, 250 aa, Serratia plymutica @
MDTGPTVTDPHFKLAPGYLEPASLPVRLALLGGPPKPDSAAFARDEEARRAALALRGSAREKLAATDAELTFPAPAKSFSCALGTDINEKKTPHLYAMMQHVLTDAGGSTYAGKNAYNRTRPFVQHDEGTCRKDMEPVLRTDGSWPSGHSAAGWAWGLVLAEVQPARATELLARGLAFGQSRVVCNAHWQSDVDAGRIMGAATVAVLHDNPAFLADLAAAKREVQDATNANLKPTEDCAAERVALSLSMH

SEQ ID NO: 105 - protein, 211 aa, Rahnella aquatilis
MEEAKPFITSQELDLTQYLPAPPADDSAQTQAELKELLQIQATRTPEQEKAAIADAQENVWRFADVMGPGFDAEKLPKTAALFERIVATEDVVDDHAKKAFNRPRPYMLDEQIHPLLKKSKSGSWPSGHSTIGYLMATVLGEMVPEKRNALFARASGYAENRLVAGFHYRSDTVMSRTGAALIAQKMEEQPDFKTEFDAAKAELRAQSGLK

SEQ ID NO: 106 - protein, 142 aa, Serratia plymutica
MDTGPTVTDPHFKLAPGYLEPASLPVRLALLGGPPKPDSAAFARDEEARRAALALRGSAREKLAATDAELTFPAPAKSFSCALGTDINEKKTPHLYAMMQHVLTDAGGSTYAGKNAYNRTRPFVQHDEGTCRKDMEPVLRTD

SEQ ID NO: 107 - protein, 122 aa, Rahnella aquatilis
MEEAKPFITSQELDLTQYLPAPPADDSAQTQAELKELLQIQATRTPEQEKAAIADAQENVWRFADVMGPGFDAEKLPKTAALFERIVATEDVVDDHAKKAFNRPRPYMLDEQIHPLLKKSKS

SEQ ID NO: 108 - protein, 132 aa, Flavobacterium johnsoniae
MDALLDGYLSEAEMPDSLLLLSPPPEHNSSLFDLDLEHAKKAVESKDKERFLQAARDADLSFPFAVKSFEPILGIEISETKTPKFYVLMRRVMTDAGLSTYAAKNHYKRERPFMVNNQKTCTPDQENILYKV

SEQ ID NO: 109 - protein, 143 aa, Methylobacterium ectorquens
MDSAPSLEKDKLAAAAPKGYLSEEATPNLVAILPPPPAGHSAAEAADRAVYNAARAFQGSPRWALATDDVADGGAALLQDYACVLGQRIDQASVPDLMRLLDRARIDIARATRVAKRRYRRLRPFVGNDLPICVARTAELADS

SEQ ID NO: 110 - protein, 134 aa, Acetobacter pasteurianus
MDSSLFGYTAQAQQFTLPDGRAFLPPPPQAEEPAQQADLRAFEKTRGLKDKARWKLAQNDANLNPSHVIKDFSCAAGFNLDPEKLPAMVNLLTSLAQPVEQDVSNEKDFWKRRRPFVGTNKDICTAHSDGLDNS

SEQ ID NO: 111 - protein, 101 aa, Serratia plymutica
SAAGWAWGLVLAEVQPARATELLARGLAFGQSRVVCNAHWQSDVDAGRIMGAATVAVLHDNPAFLADLAAAKREVQDATNANLKPTEDCAAERVALSLSMH

SEQ ID NO: 112 - protein, 82 aa, Rahnella aquatilis
STIGYLMATVLGEMVPEKRNALFARASGYAENRLVAGFHYRSDTVMSRTGAALIAQKMEEQPDFKTEFDAAKAELRAQSGLK

SEQ ID NO: 113 - protein, 77 aa, Flavobacterium johnsoniae
AVGWAWSLVLIKLFPDKQEEILKRGHDFGESRVICNAHWYSDVEMGRVMGRAAVECLCVNSAFLSDLEEVKKEMAGA

SEQ ID NO: 114 - protein, 102 aa, Methylobacterium ectorquens
SQGWAYGLIMANLMPEKATQFLVRSRLYGESRVVCGVHWLSDIEAARTGASALVAVLLADPGFRTDLERARTDLKRALSGEGAKPDPALCAREDAAARQPLL

SEQ ID NO: 115 - protein, 98 aa, Acetobacter pasteurianus
TWGWLTASILASALPDRATQIMQRGRIFGESRIVCGVHWKSDVQAGYMNGSAIFAALQEQPTFTEQMAKVRQELLALRDAKTAPDAKTCAVEQQAAQD

SEQ ID NO: 116 - protein, 253 aa, Artificial Sequence
  variant b
MDSAPSLEKDKLAAAAPKGYLSEEATPNLVAILPPPPAGHSAAEAADRAVYNAARAFQGSPRWALATDDVADGGAALLQDYACVLGQRIDQASVPDLMRLLDRARIDIARATRVAKRRYRRLRPFVGNDLPICVARTAELADSASYPSGHASQGWAYGLIMANLMPEKATQFLVRSRLYGESRVVCGVHWLSDIEAARTGASALVAVLLADPGFRTDLERARTDLKRALSGEGAKPDPALCAREDAAARQPLL

SEQ ID NO: 117 - protein, 253 aa, Artificial Sequence
  variant c
MDSAPSLEKDKLAAAAPKGYLSEEATPNLVAILPPPPAGHSAAEAADRAVYNAARAFQGSPRWALATDDVADGGAALLQDYACVLGQRIDQASVPDLMRLLDRARIDIARATRVAKRRYRRLRPFVGNDLPICVARTAELADSGSYPSGHASQGWAYGLIMANLMPEKATQFLVRSRLYGESRVVCGVHWLSDIEAARTGASALVAVLLADPGFRTDLERARTDLKRALSGEGAKPDPALCAREDAAARQPLL

SEQ ID NO: 118 - protein, 253 aa, Artificial Sequence
  variant d
MDSAPSLEKDKLAAAAPKGYLSEEATPNLVAILPPPPAGHSAAEAADRAVYNAARAFQGSPRWALATDDVADGGAALLQDYACVLGQRIDQASVPDLMRLLDRARIDIARATRVAKRRYRRLRPFVGNDLPICVARTAELADSFSYPSGHDSQGWAYGLIMANLMPEKATQFLVRSRLYGESRVVCGVHWLSDIEAARTGASALVAVLLADPGFRTDLERARTDLKRALSGEGAKPDPALCAREDAAARQPLL

SEQ ID NO: 119 - protein, 253 aa, Artificial Sequence
  variant e
MDSAPSLEKDKLAAAAPKGYLSEEATPNLVAILPPPPAGHSAAEAADRAVYNAARAFQGSPRWALATDDVADGGAALLQDYACVLGQRIDQASVPDLMRLLDRARIDIARATRVAKRRYRRLRPFVGNDLPICVARTAELADSFSYPSGHTSQGWAYGLIMANLMPEKATQFLVRSRLYGESRVVCGVHWLSDIEAARTGASALVAVLLADPGFRTDLERARTDLKRALSGEGAKPDPALCAREDAAARQPLL

SEQ ID NO: 120 - protein, 253 aa, Artificial Sequence
  variant f
MDSAPSLEKDKLAAAAPKGYLSEEATPNLVAILPPPPAGHSAAEAADRAVYNAARAFQGSPRWALATDDVADGGAALLQDYACVLGQRIDQASVPDLMRLLDRARIDIARATRVAKRRYRRLRPFVGNDLPICVARTAELADSFSYPSGHSSQGWAYGLIMANLMPEKATQFLVRSRLYGESRVVCGVHWLSDIEAARTGASALVAVLLADPGFRTDLERARTDLKRALSGEGAKPDPALCAREDAAARQPLL

SEQ ID NO: 121 - protein, 253 aa, Artificial Sequence
  variant g
MDSAPSLEKDKLAAAAPKGYLSEEATPNLVAILPPPPAGHSAAEAADRAVYNAARAFQGSPRWALATDDVADGGAALLQDYACVLGQRIDQASVPDLMRLLDRARIDIARATRVAKRRYRRLRPFVGNDLPICVARTAELADSGSYPSGHSSQGWAYGLIMANLMPEKATQFLVRSRLYGESRVVCGVHWLSDIEAARTGASALVAVLLADPGFRTDLERARTDLKRALSGEGAKPDPALCAREDAAARQPLL

SEQ ID NO: 122 - DNA, 750 bp, Serratia plymutica
atggataccgggccgacggtaacggacccgcattttaagctggcgccgggttaccttgaaccggccagcctgccggtgcgcttggccctgctcggggggccgccaaaaccggactctgcggcattcgcacgcgatgaagaggcgcggcgggcggcactggcgttacgcgggtctgcccgtgaaaaactggccgcaacggatgccgagttgaccttccctgcaccggcaaaatcgttctcgtgcgcgctgggcaccgatatcaacgagaaaaagacgccgcacctttacgccatgatgcagcacgtactgacggatgcgggtggttcaacctatgcaggaaagaacgcctataaccgtactcgcccgtttgtgcagcatgacgagggaacctgtcgcaaagacatggagccggtgctacgtaccgacggctcctggccgtcgggccactcggcggcaggctgggcctggggattggtgctggccgaggtgcagccggcacgggcgaccgagctgctggcacgcggcttggcctttggtcagagccgtgtggtctgcaatgcgcattggcagagcgacgtggatgcagggcgcatcatgggggctgccacggtcgccgtgctgcatgataacccggctttcctggcagatttggccgccgccaagcgtgaagtgcaggacgctaccaacgcgaacctgaaacccaccgaggactgcgcggctgaacgtgttgcactctctttgagtatgcac

SEQ ID NO: 123 - DNA, 633 bp, Rahnella aquatilis
atggaagaagccaaaccctttatcaccagtcaggaactggatctgacccaatatctgccagcgccaccggcggatgattcggcgcagacccaagcggagctgaaagaattgctccaaattcaggccacccgcacgccggagcaggaaaaagcggcgattgctgatgcgcaagaaaacgtctggcgttttgccgatgtgatggggccgggctttgatgccgagaaactgccgaaaaccgccgcgctgtttgagcgtattgtggcgacagaagacgtggtggacgatcacgccaagaaagcgtttaaccgtccgcgtccttatatgctggatgaacaaattcatccgctgctgaaaaagtctaaatccggttcatggccttccggtcattccaccatcggttatctgatggcgacggtgctgggcgaaatggtgccggaaaaacgcaatgcgctgtttgcccgtgcatccggttatgccgaaaaccgtctggtggctggtttccattaccgttctgataccgtcatgagccgcaccggtgccgcgctgattgctcagaaaatggaagaacagccagatttcaaaaccgaattcgacgcggcgaaagcggaacttcgcgcccaatctggcctgaaa

SEQ ID NO: 124 - DNA, 36 bp, Artificial Sequence
  primer124
gctgccaccatggataccgggccgacggtaacggac

SEQ ID NO: 125 - DNA, 34 bp, Artificial Sequence
  primer125
gatcaagcttgtgcatactcaaagagagtgcaac

SEQ ID NO: 126 - DNA, 36 bp, Artificial Sequence
  primer126
gttcaggccatggaagaagccaaaccctttatcacc

SEQ ID NO: 127 - DNA, 34 bp, Artificial Sequence
  primer 127
acgaaagctttttcaggccagattgggcgcgaag

SEQ ID NO: 128 - DNA, 759 bp, Artificial Sequence
  DNA coding phosphatase
atggacagtgccccgagtctggaaaaagataaactggcggccgcagctccgaaaggctatctgagcgaagaagctaccccgaacctggttgcaattctgccgccgccgccggcaggtcattctgcagccgaagcagctgatcgtgccgtctataatgcgacccgcgcctttcagggtagcccgcgttgggcactggcaacggatgacgtggcagatggtggtgcagcactgctggaagactacgcttgcgtgctgggtcagcgcatcgatcaagcaaacgttccggacctgatgcgtctgctggatcgtgcacgcattgacatcgcacgtgctacccgcgttgcgaaacgtcgctatcgtcgcctgcgcccgtttgttggtaatgatctgccgatttgtgtcgcacgtaccgctgaactggcggacagcttctcttatccgagtggtcatgcgtcccagggttgggcctacggtctgatcatggcaaacctgatgccggaaaaagccacgcaactgctggtccgttcacgcctgtacggcgaatcgcgcgtggtttgcggtgtgcactggctgagcgatattgaagcagctcgtaccggtgcatctgccctggtcgccgtgctgctggcagatccgagtttccgcacggacctggaacgtgcacgcgctgatctgaaacgtgcgctgtccggcgaaggtgcaaaaccggacccggctctgtgtgcccgtgaagatgctgctgcccgtcaaccgctgctg

SEQ ID NO: 129 - DNA, 34 bp, Artificial Sequence
  primer(5)
ggcgatggccatggacagtgccccgagtctggaa

SEQ ID NO: 130 - DNA, 32 bp, Artificial Sequence
  primer(6)
taaagaagcttcagcagcggttgacgggcagc

SEQ ID NO: 131 - DNA, 33 bp, Artificial Sequence
  primer(7)
actcggatagctagcgctgtccgccagttcagc

SEQ ID NO: 132 - DNA, 39 bp, Artificial Sequence
  primer(8)
gcggacagcgctagctatccgagtggtcatgcgtcccag

SEQ ID NO: 133 - DNA, 31 bp, Artificial Sequence
  primer(9)
aataggatccgctgtccgccagttcagcggt

SEQ ID NO: 134 - DNA, 39 bp, Artificial Sequence
  primer(10)
gacagaggatcctatccgagtggtcatgcgtcccagggt

SEQ ID NO: 135 - DNA, 39 bp, Artificial Sequence
  primer(11)
gatcagaccatatgcccaaccctgggagtcatgaccact

SEQ ID NO: 136 - DNA, 33 bp, Artificial Sequence
  primer(12)
cagggttgggcatatggtctgatcatggcaaac

SEQ ID NO: 137 - DNA, 39 bp, Artificial Sequence
  primer(13)
gatcagaccatatgcccaaccctgggacgtatgaccact

SEQ ID NO: 138 - DNA, 39 bp, Artificial Sequence
  primer(14)
gatcagaccatatgcccaaccctgggagctatgaccact

SEQ ID NO: 139 - DNA, 39 bp, Artificial Sequence
  primer(15)
gacagaggatcctatccgagtggtcattcgtcccagggt